(19) United States
(12) Patent Application Publication
(10) Pub. No.: US 2004/0171073 A1
(43) Pub. Date: Sep. 2, 2004

(54) Compounds for modulation of cholesterol transport

(75) Inventors: Thomas J.F. Neiland, Somerville, MA (US); Monty Krieger, Needham, MA (US); Tomas Kirchausen, Brighton, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignees: Massachusetts Institute of Technology; Center for Blood Research, Inc.

(21) Appl. No.: 10/681,746

(22) Filed: Oct. 8, 2003

Related U.S. Application Data
(60) Provisional application No. 60/417,083, filed on Oct. 8, 2002.

(51) Int. Cl. G01N 33/53; G01N 33/543

(52) U.S. Cl. 435/7.1; 436/518; 530/324

(57) ABSTRACT

Methods for regulation of lipid and cholesterol uptake are described which are based on regulation of the expression or function of the SR-BI HDL receptor. The examples demonstrate that estrogen dramatically downregulates SR-BI under conditions of tremendous upregulation of the LDL-receptor. The examples also demonstrate the upregulation of SR-BI in rat adrenal membranes and other non-placental steroidogenic tissues from animals treated with estrogen, but not in other non-placental non-steroidogenic tissues, including lung, liver, and skin. Examples further demonstrate the uptake of fluorescently labeled HDL into the liver cells of animal, which does not occur when the animals are treated with estrogen. Examples also demonstrate the in vivo effects of SR-BI expression on HDL metabolism, in mice transiently overexpressing hepatic SR-BI following recombinant adenovirus infection. Overexpression of the SR-BI in the hepatic tissue caused a dramatic decrease in cholesterol blood levels. These results demonstrate that modulation of SR-BI levels, either directly or indirectly, can be used to modulate levels of cholesterol in the blood.

### CROSS REFERENCE TO RELATED APPLICATIONS

This application claims priority to U.S. Provisional Application Serial No. 60/417,083 filed on Oct. 8, 2002.

The U.S. government has certain rights to this invention by virtue of grants HL52212, HL 66105 and HL64737 from the National Institutes of Health-National Heart, Lung and Blood Institute.

### FIELD OF THE INVENTION

The present invention is generally in the area of compounds for modulation of cholesterol transport and lipid regulation mediated via the SR-BI scavenger receptor.

### BACKGROUND OF THE INVENTION

The intercellular transport of lipids through the circulatory system requires the packaging of these hydrophobic molecules into water-soluble carriers, called lipoproteins, and the regulated targeting of these lipoproteins to appropriate tissues by receptor-mediated pathways. The most well characterized lipoprotein receptor is the LDL receptor, which binds to apolipoproteins B-100 (apoB-100), and E (apoE), which are constituents of low density lipoprotein (LDL), the principal cholesteryl-ester transporter in human plasma, very low-density lipoprotein (VLDL), a triglyceride-rich carrier synthesized by the liver, intermediate-density lipoprotein (IDL), and catabolized chylomicrons (dietary triglyceride-rich carriers). Kreiger, et al., in WO96/00288 “Class BI and CI Scavenger Receptors” by Massachusetts Institute of Technology, U.S. Pat. Nos. 6,359,859 and 6,429,289 (“Krieger, et al.”) characterized and cloned hamster and murine homologs of SR-BI, an AcLDL and LDL binding scavenger receptor. It was reported by Kreiger, et al. that the SR-BI receptor is expressed principally in steroidogenic tissues and liver and appears to mediate HDL-transfer and uptake of cholesterol. Competitive binding studies show that SR-BI binds LDL, modified LDL, negatively charged phospholipid, and HDL. Direct binding studies show that SR-BI expressed in mammalian cells (for example, a variant of CHO cells) binds HDL, without cellular degradation of the HDL-apoprotein, and lipid is accumulated within cells expressing the receptor. These studies indicated that SR-BI might play a major role in transfer of cholesterol from peripheral tissues, via HDL, into the liver and steroidogenic tissues, and that increased or decreased expression in the liver or other tissues may be useful in regulating uptake of cholesterol by cells expressing SR-BI, thereby decreasing levels in foam cells and deposition at sites involved in atherogenesis.

Subsequent studies confirmed that SR-BI not only binds to lipid, but also transfers cholesterol into and out of cells, as described in U.S. Pat. Nos. 5,962,322 and 5,925,333 to Krieger, et al. Moreover, SR-BI is preferentially expressed in steroidogenic tissues, and plays a role in lipid regulation, affecting not only cholesterol levels but also female fertility, as described by WO99/11288 by Massachusetts Institute of Technology.

The role of SR-BI in cholesterol uptake and transfer can be manipulated via SR-BI, for example, as demonstrated using probucol treatment to restore female fertility, as described by Miettinen, et al. (2001) J. Clin. Invest. 108(11): 1717-1722. This work clearly demonstrates that there is a need for additional drugs that that stimulate or inhibit SR-BI mediated lipid uptake and metabolism.

It is an object of the present invention to provide drugs and methods and reagents for designing drugs, that can stimulate or inhibit the binding to and lipid movements mediated by SR-BI and redirect uptake and metabolism of lipids and cholesterol by cells.

### SUMMARY OF THE INVENTION

Compounds for regulation of cholesterol transport are described which are based on regulation of the expression or function of SR-BI. SR-BI mediates both selective uptake of lipids, mainly cholesterol esters, from HDL to cells and efflux of cholesterol from cells to lipoproteins. The mechanism underlying these lipid transfers is distinct from classic receptor mediated endocytosis, but remains poorly understood. To investigate SR-BI's mechanism of action and in vivo function, a high throughput screen was developed to identify small molecule inhibitors of SR-BI-mediated lipid transfer in intact cells. Two hundred compounds were identified that block lipid transport (BLTs), both selective uptake and efflux, in the low nanomolar to micromolar range. The effects of these compounds were highly specific to the SR-BI pathway, because they did not interfere with clathrin-based receptor-mediated endocytosis or with other forms of intracellular vesicular traffic. As demonstrated by the examples, five BLTs (BLT-1 [MIT 9952-53]; BLT-2 [MIT 9952-61]; BLT-3 [MIT 9952-19]; BLT-4 [MIT 9952-29]; and BLT-5 [MIT 9952-6]) enhanced, rather than inhibited, HDL binding by increasing SR-BI's binding affinity for HDL (decreased dissociation rates). Others inhibited HDL binding. These should be useful in the management of atherosclerosis, treatment of infertility, or conversely, as contraceptives and in the treatment of Tangier's disease.

### BRIEF DESCRIPTION OF THE DRAWINGS

FIGS. 1A-1C are graphs of the concentration dependence of the inhibition by BLTs of SR-BI-mediated lipid transfer between HDL and cells. ldlA[mSR-BI] cells were incubated with the indicated concentrations of BLTs and their effects on (A) DiI uptake from DiI-HDL, (B) [³H]CE uptake from [³H]CE-HDL and (C) the efflux of [³H]cholesterol from cells to HDL were determined. The 100% of control values were: A, 50.6 ng HDL protein equivalents/well (384-well plates) and B, 3908 ng HDL protein equivalents/mg cellular protein. In C, the data were normalized such that the maximum amount of [³H]cholesterol transferred from cells to HDL in the absence of compounds (55.7% of total) was set to 100%. The 0% value corresponds to the efflux of [3H] cholesterol transferred from ldlA[mSR-BI] cells to HDL without BLTs and in the presence of saturating inhibitory amounts of the specific anti-SR-BI blocking monoclonal antibody KKB-1 (15% of total). The efflux of [³H]cholesterol from ldlA-7 cells measured in the absence or presence of KKBI was 15% and 10% of total cellular [³H]cholesterol, respectively.

FIGS. 2A-2D are graphs of cell surface expression of SR-BI. ldlA[mSR-BI] and ldlA-7 cells were treated for 3 hrs with or without BLTs at their corresponding IC_{CE}95 concentrations (1 μM for BLT-1 (MIT 9952-53) and BLT-2 (MIT 9952-61), 50 μM for BLT-3 (MIT 9952-19), BLT-4 (MIT 9952-29) and BLT-5 (MIT 9952-6)) followed by determination of surface expression levels of SR-BI by flow cytometry. Panels A-C show histograms of the surface expression for ldlA[mSR-BI] cells without BLTs, ldlA[mSR-BI] cells with 1 μM BLT-1 (MIT 9952-53), and ldlA-7 cells without BLTs, respectively. Panel D summarizes the results in ldlA[mSR-BI] cells for all five BLTs, with the value determined without compounds set to 100%. n, number of independent determinations; SD, standard deviation.

FIGS. 3A-3E shows the effects of BLTs on SR-BI-mediated cholesterol ether uptake from HDL, cellular cholesterol efflux to HDL and HDL binding. The effects of indicated concentrations of BLTs (panels A-E) on SR-BI-mediated uptake of [³H]CE from [³H]CE-HDL (solid lines, no symbols), efflux of [³H]cholesterol from cells to HDL (dashed lines), or binding of ¹²⁵I-HDL to cells (solid lines, filled symbols) were determined using ldlA[mSR-BI] cells. To simplify comparisons, the lowest observed [³H]CE uptake and [³H]cholesterol efflux values (from FIG. 2) were compared as 0% and the values in the absence of BLTs as 100%. The 100% control value for the ¹²⁵I-HDL binding in the absence of BLTs was 403 ng HDL protein/mg cell protein.

FIG. 4 is a graph of the effects of BLT-1 (MIT 9952-53) on the concentration dependence of ¹²⁵I-HDL binding to ldlA[mSR-BI] cells. The binding of ¹²⁵I-HDL to ldlA[mSR-BI] cells was determined in duplicate at the indicated concentrations of HDL in the presence (blue) or absence (black) of 1 μM BLT-1 (MIT 9952-53; IC_{CE}95). Each value was corrected for binding of ¹²⁵I-HDL in the presence of 40-fold excess of unlabeled HDL to ldlA [mSR-BI] cells in the presence of BLT-1 (MIT 9952-53).

### DETAILED DESCRIPTION OF THE INVENTION

I. Modulators of SR-BI Transport of Cholesterol.

Libraries of compounds have been screened using an assay such as the assays described below for alteration in HDL binding. These compounds can be proteins, DNA sequences, polysaccharides, or synthetic organic compounds. Approximately 200 that have been identified as having activity are listed below in Table I.

II. Screening of Compounds to Inhibit or Enhance SR-BI Activity.

The SR-BI proteins and antibodies and their DNAs can be used in screening of drugs which modulate the activity and/or the expression of SR-BI. The cDNA encoding SR-BI has been cloned and is reported U.S. Pat. No. 6,359,859 and 6,429,289 and is listed in GenBank. The cDNA encoding SR-BI yields a predicted protein sequence of 509 amino acids. The drugs which enhance SR-BI activity should be useful in treating or preventing atherosclerosis, fat uptake by adipocytes, and some types of endocrine disorders. The drugs which inhibit SR-BI activity should be useful as contraceptives and in the treatment of Tangiers disease.

The assays described below clearly provide routine methodology by which a compound can be tested for an inhibitory effect on binding of a specific compound, such as a radiolabeled modified HDL and LDL or polyion. The in vitro studies of compounds which appear to inhibit binding selectively to the receptors can then be confirmed by animal testing. Since the molecules are so highly evolutionarily conserved, it is possible to conduct studies in laboratory animals such as mice to predict the effects in humans.

Studies based on inhibition of binding are predictive for indirect effects of alteration of receptor binding. For example, inhibition of cholesterol-HDL binding to the SR-BI receptor leads to decreased uptake by cells of cholesterol and therefore inhibits cholesterol transport by cells expressing the SR-BI receptor. Increasing cholesterol-HDL binding to cells increases removal of lipids from the blood stream and thereby decreases lipid deposition within the blood stream. Studies have been conducted using a stimulator to enhance macrophage uptake of cholesterol and thereby treat atherogenesis, using M-CSF (Schaub, et al., 1994 Arterioscler. Thromb. 14(1), 70-76; Inaba, et al., 1993 J. Clin. Invest. 92(2), 750-757).

The following assays can be used to screen for compounds which are effective in methods for alter SR-BI expression, concentration, or transport of cholesterol.

Assays for Alterations in SR-BI Binding or Expression

Northern blot analysis of murine tissues shows that SR-BI is most abundantly expressed in adrenal, ovary, liver, testes, and fat and is present at lower levels in some other tissues. SR-BI mRNA expression is induced upon differentiation of 3T3-L1 cells into adipocytes. Both SR-BI and CD36 display high affinity binding for acetylated LDL with an apparent dissociation constant in the range of approximately 5 μg protein/ml. The ligand binding specificities of CD36 and SR-BI, determined by competition assays, are similar, but not identical: both bind modified proteins (acetylated LDL, maleylated BSA), but not the broad array of other polyanions (e.g. fucoidin, polyinosinic acid, polyguanosinic acid) which are ligands of the class A receptors. SR-BI displays high affinity and saturable binding of HDL which is not accompanied by cellular degradation of the HDL. HDL inhibits binding of AcLDL to CD36, suggesting that it binds HDL, similarly to SR-BI. Native LDL, which does not compete for the binding of acetylated LDL to either class A receptors or CD36, competes for binding to SR-BI.

¹²⁵I-AcLDL Binding, Uptake and Degradation Assays.

Scavenger receptor activities at 37° C. are measured by ligand binding, uptake and degradation assays as described by Krieger, Cell 33, 413-422, 1983; and Freeman et al., (1991) Proc Natl Acad Sci USA. 1991 Jun. 1;88(11):4931-5). The values for binding and uptake are combined and are presented as binding plus uptake observed after a 5 hour incubation and are expressed as ng of ¹²⁵I-AcLDL protein per 5 hr per mg cell protein.

Degradation activity is expressed as ng of ¹²⁵I-AcLDL protein degraded in 5 hours per mg of cell protein. The specific, high affinity values represent the differences between the results obtained in the presence (single determinations) and absence (duplicate determinations) of excess unlabeled competing ligand. Cell surface 4° C. binding is assayed using either method A or method B as indicated. In method A, cells are prechilled on ice for 15 min, re-fed with ¹²⁵I-AcLDL in ice-cold medium B supplemented with 10% (v/v) fetal bovine serum, with or without 75-200 μg/ml unlabeled M-BSA, and incubated 2 hr at 4° C. on a shaker. Cells are then washed rapidly three times with Tris wash buffer (50 mM Tris-HCl, 0.15 M NaCl, pH 7.4) containing 2 mg/ml BSA, followed by two 5 min washes, and two rapid washes with Tris wash buffer without BSA. The cells are solubilized in 1 ml of 0.1 N NaOH for 20 min at room temperature on a shaker, 30 μl are removed for protein determination, and the radioactivity in the remainder is determined using a LKB gamma counter. Method B differs from method A in that the cells are prechilled for 45 minutes, the medium contains 10 mM HEPES and 5% (v/v) human lipoprotein-deficient serum rather than fetal bovine serum, and the cell-associated radioactivity released by treatment with dextran sulfate is measured as described by Krieger, (1983) Cell 33, 413-422; Freeman et al., (1991) Proc Natl Acad Sci USA. 1991 Jun. 1;88(11):4931-5)).

Northern Blot Analysis.

0.5 micrograms of poly(A)+ RNA prepared from different murine tissues or from 3T3-L1 cells on zero, two, four, six or eight days after initiation of differentiation into adipocytes as described by Baldini et al., 1992 Proc. Natl. Acad. Sci. U.S.A. 89, 5049-5052, is fractionated on a formaldehyde/agarose gel (1.0%) and then blotted and fixed onto a Biotrans™ nylon membrane. The blots are hybridized with probes that are ³²P-labeled (2×10⁶ dpm/ml, random-primed labeling system). The hybridization and washing conditions, at 42° C. and 50° C., respectively, are performed as described by Charron et al., 1989 Proc. Natl. Acad. Sci. U.S.A. 86, 2535-2539. The probe for SR-BI mRNA analysis was a 0.6 kb BamHI fragment from the cDNAs coding region. The coding region of murine cytosolic hsp70 gene (Hunt and Calderwood, 1990 Gene 87, 199-204) is used as a control probe for equal mRNA loading.

SR-BI protein in tissues is detected by blotting with polyclonal antibodies to SR-BI.

HDL Binding Studies

HDL and VLDL binding to SR-BI and CD36 are conducted as described for LDL and modified LDL.

Studies conducted to determine if the HDL which is bound to SR-BI is degraded or recycled and if lipid which is bound to the HDL is transferred into the cells are conducted using fluorescent lipid-labeled HDL, ³H-cholesteryl ester labeled HDL and ¹²⁵I-HDL added to cultures of transfected or untransfected cells at a single concentration (10 μg protein/ml). HDL associated with the cells is measured over time. A steady state is reached in approximately thirty minutes to one hour. A fluorescent ligand, DiI, or ³H-cholesterol ester is used as a marker for lipid (for example, cholesterol or cholesterol ester) uptake by the cell. Increasing concentration of DiI indicates that lipid is being transferred from the HDL to the receptor, then being internalized by the cell. The DiI-depleted HDL is then released and replaced by another HDL molecule.

HDL Binding to SR-BI

Competition binding studies demonstrate that HDL and VLDL (400 μg/ml) competitively inhibit binding of ¹²⁵I-AcLDL to SR-BI. Direct binding of ¹²⁵I-HDL to cells expressing SR-BI is also determined.

Tissue distribution of SR-BI

To explore the physiological functions of SR-BI, the tissue distribution of SR-BI was determined in murine tissues, both in control animals and estrogen treated animals, as described in the following examples. Each lane is loaded with 0.5 μg of poly(A)+ RNA prepared from various murine tissues: kidney, liver, adrenals, ovaries, brain, testis, fat, diaphragm, heart, lung, spleen, or other tissue. The blots are hybridized with a 750 base pair fragment of the coding region of SR-BI. SR-BI mRNA is most highly expressed in adrenals, ovary and liver is moderately or highly expressed in fat depended on the source and is expressed at lower levels in other tissues. Blots using polyclonal antibodies to a cytoplasmic region of SR-BI demonstrate that very high levels of protein are present in liver, adrenal tissues, and ovary in mice and rats, but only very low or undetectable levels are present in either white or brown fat, muscle or a variety of other tissues. Bands in the rat tissues were present at approximately 82 kD. In the mouse tissues, the 82 kD form observed in the liver and steroidogenic tissues is the same size observed in SR-BI-transfected cultured cells.

Assays for testing compounds for useful activity can be based solely on interaction with the receptor protein, preferably expressed on the surface of transfected cells such as those described above, although proteins in solution or immobilized on inert substrates can also be utilized, where the indication is inhibition or increase in binding of lipoproteins.

Alternatively, the assays can be based on interaction with the gene sequence encoding the receptor protein, preferably the regulatory sequences directing expression of the receptor protein. For example, antisense which binds to the regulatory sequences, and/or to the protein encoding sequences can be synthesized using standard oligonucleotide synthetic chemistry. The antisense can be stabilized for pharmaceutical use using standard methodology (encapsulation in a liposome or microsphere; introduction of modified nucleotides that are resistant to degradation or groups which increase resistance to endonucleases, such as phosphorothiodates and methylation), then screened initially for alteration of receptor activity in transfected or naturally occurring cells which express the receptor, then in vivo in laboratory animals. Typically, the antisense would inhibit expression. However, sequences which block those sequences which “turn off” synthesis can also be targeted.

II. Methods of Regulation of SR-BI Cholesterol Transport.

The HDL receptor SR-BI plays an important role in controlling the structure and metabolism of HDL (Acton, et al. (1996) Science 271, 518-20; Krieger, M. (1999) Annu Rev Biochem 68, 523-58). Studies in mice have shown that alterations in SR-BI expression can profoundly influence several physiologic systems, including those involved in biliary cholesterol secretion, female fertility, red blood cell development, atherosclerosis and the development of coronary heart disease (Trigatti, et al. (1999) Pro. Nat. Acad. Sci. USA 96, 9322-7; Kozarsky, et al. (2000) Arterio. Thromb. Vasc. Biol. 20, 721-7; Arai, et al. (1999) J. Biol. Chem. 274, 2366-71; Holm, et al. (2002) Blood 99, 1817-24; Miettinen, et al. (2001) J. Clin. Invest. 108, 1717-22; Ueda, et al. (2000) J. Biol. Chem. 275, 20368-73; Kozarsky, et al. (1997) Nature 387, 414-7; Braun, et al. (2002) Cir. Res. 90, 270-276; Mardones, et al. (2001) J. Lipid Res. 42, 170-180)) SR-BI controls HDL metabolism by mediating the cellular selective uptake of cholesteryl esters and other lipids from plasma HDL. During selective uptake (Glass, et al. (1983) Proc. Nat. Acad. Sci. USA 80, 5435-9; Glass, et al. (1985) J. Biol. Chem. 260, 744-50; Stein, et al. (1983) Biochimica et Biophysica Acta 752, 98-105), HDL binds to SR-BI and its lipids, primarily neutral lipids such as cholesteryl esters in the core of the particles, are transferred to the cells. The lipid-depleted particles are subsequently released back into the extracellular space. Although the mechanism of SR-BI-mediated selective lipid uptake and the subsequent intracellular transport of these lipids has only just begun to be explored (Krieger 1999; Krieger, M. (2001) J Clin Invest 108, 793-7; Uittenbogaard, et al. (2002) J. Biol. Chem. 277, 4925-4931), it is clearly fundamentally different from the pathway of receptor-mediated endocytosis via clathrin-coated pits and vesicles used by the low-density lipoprotein (LDL) receptor to deliver cholesterol esters from LDL to cells (Brown, M. S. & Goldstein, J. L. (1986) Science 232, 34-47). SR-BI can also mediate cholesterol efflux from cells to HDL (Temel, et al. (2002) J Biol Chem 8, 8).

It has now been demonstrated that SR-BI plays critical roles in HDL lipid metabolism and cholesterol transport. SR-BI appears to be responsible for cholesterol delivery to steroidogenic tissues and liver, and actually transfers cholesterol from HDL particles through the liver cells and into the bile canniculi, where it is passed out into the intestine. Data indicates that SR-BI is also expressed in the intestinal mucosa. It would be useful to increase expression of SR-BI in cells in which uptake of cholesterol can be increased, freeing HDL to serve as a means for removal of cholesterol from storage cells such as foam cells where it can play a role in atherogenesis.

Compounds which alter receptor protein binding are preferably administered in a pharmaceutically acceptable vehicle. Suitable pharmaceutical vehicles are known to those skilled in the art. For parenteral administration, the compound will usually be dissolved or suspended in sterile water, phosphate buffered saline, or saline. For enteral administration, the compound will be incorporated into an inert carrier in tablet, liquid, or capsular form. Suitable carriers may be starches or sugars and include lubricants, flavorings, binders, and other materials of the same nature. The compounds can also be administered locally by topical application of a solution, cream, gel, or polymeric material (for example, a Pluronic™, BASF). The compounds may also be formulated for sustained or delayed release.

Alternatively, the compound may be administered in liposomes or microspheres (or microparticles). Methods for preparing liposomes and microspheres for administration to a patient are known to those skilled in the art. U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is by G. Gregoriadis, Chapter 14. “Liposomes”, Drug Carriers in Biology and Medicine pp. 287-341 (Academic Press, 1979). Microspheres formed of polymers or proteins are well known to those skilled in the art, and can be tailored for passage through the gastrointestinal tract directly into the bloodstream. Alternatively, the compound can be incorporated and the microspheres, or composite of microspheres, implanted for slow release over a period of time, ranging from days to months. See, for example, U.S. Pat. Nos. 4,906,474, 4,925,673, and 3,625,214.

The present invention will be further understood by reference to the following non-limiting examples.

### EXAMPLE 1

### Identification of Chemical Inhibitors of the Selective Transfer of Lipids mediated by the HDL Receptor SR-BI

Abbreviations

| | |
|---|---|
| HDL | High Density Lipoprotein |
| mSR-BI | Murine Scavenger Receptor, class B, type I |
| LDL | Low Density Lipoprotein |
| BLT | Block Lipid Transfer |
| DiI | 1′-dioctadecyl-3,3,3′,3′- |
| | tetramethylindocarbocyanine perchlorate |
| CE | Cholesteryl ether |
| DMSO | Dimethylsulfoxide |
| PBS | Phosphate Buffered Saline |
| EGF | Epidermal Growth Factor |
| VSV-G | Vesicular Stomatitis Virus Glycoprotein |
| EGFP | enhanced Green Fluorescent Protein |
| IC | Inhibitory Concentration |
| EC | Effective Concentration |
| ACTH | Adrenocorticotropic Hormone |
| FC | Free cholesterol |

A high-throughput screen of a chemical library to identify potent small molecule inhibitors of SR-BI-mediated lipid transport. Five chemicals that block lipid transport, BLT-1-BLT-5 (BLT-1 corresponds to MIT 9952-53; BLT-2 corresponds to MIT 9952-61; BLT-3 corresponds to MIT 9952-19; BLT-4 corresponds to MIT 9952-29; and BLT-5 corresponds to MIT 9952-6), were tested and their effects on SR-BI activity in cultured cells. All five inhibited SR-BI-mediated selective lipid uptake from HDL and efflux of cellular cholesterol to HDL. One of these, BLT-1, was particularly potent, inhibiting lipid transport in the low nanomolar concentration range. Unexpectedly, all five BLTs enhanced HDL binding to SR-BI by increasing the binding affinity.

Methods

Lipoproteins and Cells

Human HDL was isolated and labeled with either ¹²⁵I (¹²⁵I-HDL), 1,1′-dioctadecyl-3,3,3′,3′-tetramethylindocarbocyanine perchlorate (DiI, Molecular Probes; DiI-HDL) or [³H]cholesteryl oleyl ether ([³H]CE, [³H]CE-HDL) (Gu, et al. (1998) J.

Biol. Chem. 273, 26338-48; Gu, et al. (2000) J. Biol. Chem. 275, 29993-30001; Acton, et al. (1994) J. Biol. Chem. 269, 21003-9; Pitas, et al. (1981) Arteriosclerosis 1, 177-85). LDL receptor deficient Chinese hamster ovary cells that express low levels of endogenous SR-BI, ldlA-7 (Kingsley, et al. (1984) Proc. Nat. Acad. Sci. USA 81, 5454-8), ldlA-7 cells stably transfected to express high levels of murine SR-BI (ldlA[mSR-BI])(Acton, et al., 1996), Y1-BS1 murine adrenocortical cells that express high levels of SR-BI after induction with ACTH (Rigotti, et al. (1996) J. Biol. Chem. 271, 33545-9), monkey kidney BS-C1 cells (Kapoor, et al. (2000) Journal of Cell Biology 150, 975-88) and HeLa cells (Temel, et al. (2002) J Biol Chem 8, 8) were maintained as previously described.

High Throughput Screen

On day 0, ldlA[mSR-BI] cells were plated at 15,000 cells/well in clear bottom, black wall 384-well black assay plates (Costar) in 50 μl of medium A (Ham's F12 supplemented with 2 mM L-glutamine, 50 units/ml penicillin/50 μg/ml streptomycin, and 0.25 mg/ml G418.) supplemented with 10% fetal bovine serum (medium B). On day 1, cells were washed once with medium C (medium A with 1% (w/v) bovine serum albumin (BSA) and 25 mM HEPES pH 7.4, but no G418) and refed with 40 μl of medium C.

Compounds (16,320 from the DiverSet E, Chembridge Corp.) dissolved in 100% DMSO were individually robotically ‘pin’ transferred (40 nl) (http://iccb.med.harvard.edu) to the wells to give a nominal concentration of 10 μM (0.01% DMSO). After an 1 hr incubation at 37° C., DiI-HDL (final concentration of 10 μg protein/ml) in 20 μl of medium C was added. Two hours later, fluorescence was measured at room temperature (approximately 2 minutes/plate) using a Analyst plate reader (Rhodamine B dichroic filter, emission 525 nm and excitation 580 nm; LJL Biosystems), both prior to removing the incubation medium (to test for autofluorescence and quenching) and after the medium removal and four washes with 80 μl of PBS/1 mM MgCl₂/0.1 mM CaCl₂ to determine cellular uptake of DiI. All compounds were sampled in duplicate on different plates, and each screen included ldlA-7 and ldlA[mSR-BI] cells in the presence and/or absence of a 40-fold excess of unlabeled HDL, but with no added compounds, as controls.

Assays

For the assays, all media and buffers contained 0.5% DMSO and 0.5% bovine serum albumin to maintain compound solubility. Cells were pre-incubated with BLTs for 1 hr (or 2.5 hrs for transferrin, EGF and cholera toxin uptake experiments) and all the experiments were performed at 37° C. Detailed characterization of the BLTs and their effects was performed with compounds whose identities and purities were confirmed by LC-MS.

(i) Lipid Uptake from HDL, Cholesterol Efflux to HDL and HDL Binding Assays.

Assays for the uptake of lipids from DiI-HDL and [³]CE-HDL, efflux of [³H]cholesterol from labeled cells, and ¹²⁵I-HDL binding were performed as described by Acton et al. Science (1996) January 26;271(5248):518-20; Gu, et al. J Biol. Chem. (2000) September 29;275(39):29993-30001; and Ji, et al., J. Biol. Chem. (1997) 272, 20982-5. In some experiments, values were normalized so that the 100% of control represents activity in the absence of compounds and 0% represents activity determined in the presence of a 40-fold excess of unlabeled HDL or, for Y1-BS1 cells, in the presence of a 1:500 dilution of the KKB-1 blocking antibody (Gu, et al., 2000, generous gift from Karen Kozarsky). The amounts of cell-associated [³H]cholesteryl ether are expressed as the equivalent amount of [³H]CE-HDL protein (ng) to permit direct comparison of the relative amounts of ¹²⁵I-HDL binding and [³H]CE uptake.

The rates of HDL dissociation from cells were determined by incubation of the cells with ¹²⁵I-HDL (10 μg protein/ml, 2 hrs, 37° C.) with and without BLTs. The medium was then either replaced with the same medium in which the ¹²⁵I-HDL was substituted by a 40-fold excess of unlabeled HDL or a 40-fold excess of unlabeled HDL was added to the labeled incubation medium. The amounts of cell-associated ¹²⁵I-HDL were then determined as a function of time. The two methods gave similar results.

(ii) Fluorescence Microscopic Analysis of Intracellular Trafficking and Cytoskeletal Organization.

Receptor mediated endocytosis of Alexa-594 labeled transferrin or FITC labeled epidermal growth factor (EGF, Molecular Probes) by HeLa cells (Spiro, et al. (1996) Mol Biol Cell 7, 355-67) and uptake of Alexa-594-labeled holo-cholera toxin (kind gift of Dr Wayne Lencer, Childrens Hospital, HMS) by BSC-1 cells were detected by fluorescent microscopy. The intracellular transport of the temperature sensitive glycoprotein of vesicular stomatitis virus (VSVG^{ts045}) fused at its carboxyl terminus to EGFP (VSVG^{ts045}-EGFP) from the endoplasmic reticulum to the plasma membrane, after a shift from 40° C. to 32° C. for 2 hrs, was determined by fluorescent microscopy. The effects of the compounds on the distribution of actin using rhodamine labeled phalloidin and tubulin using the FITC labeled DM1 α monoclonal antibody (Sigma Co.) in ldlA[mSR-BI] cells were determined as described by Rigotti, et al. (1996) J. Biol. Chem. 271, 33545-9 by fluorescence microscopy using an air 63× objective (Nikon).

(iii) Flow Cytometric Analysis of SR-BI Cell Surface Expression.

Cells were incubated for 3 hrs (medium C) with or without BLTs at their IC_{CE}95 concentrations, harvested with PBS containing 2 mM EDTA and compounds, and the levels of SR-BI surface expression in unfixed cells were determined by flow cytometry with the KKB-1 antibody (Gu, et al. (1998) J. Biol. Chem. 273, 26338-48).

Results

High-Throughput Screening for Inhibitors of SR-BI-Mediated Selective Lipid Uptake.

Cellular uptake and accumulation of the fluorescent lipophilic dye DiI from DiI-labeled HDL (DiI-HDL) is a reliable surrogate of SR-BI-dependent selective uptake of the cholesteryl esters in HDL. To identify small molecule inhibitors of SR-BI-mediated selective lipid uptake, 16,320 compounds representing the DiverSet E of the Chembridge library collection were screened for their abilities to block the cellular uptake of DiI from DiI-HDL. The compounds were tested at a nominal concentration of 10 micromolar in a 384-well-plate assay using ldlA[mSR-BI] cells that express a high level of mSR-BI.

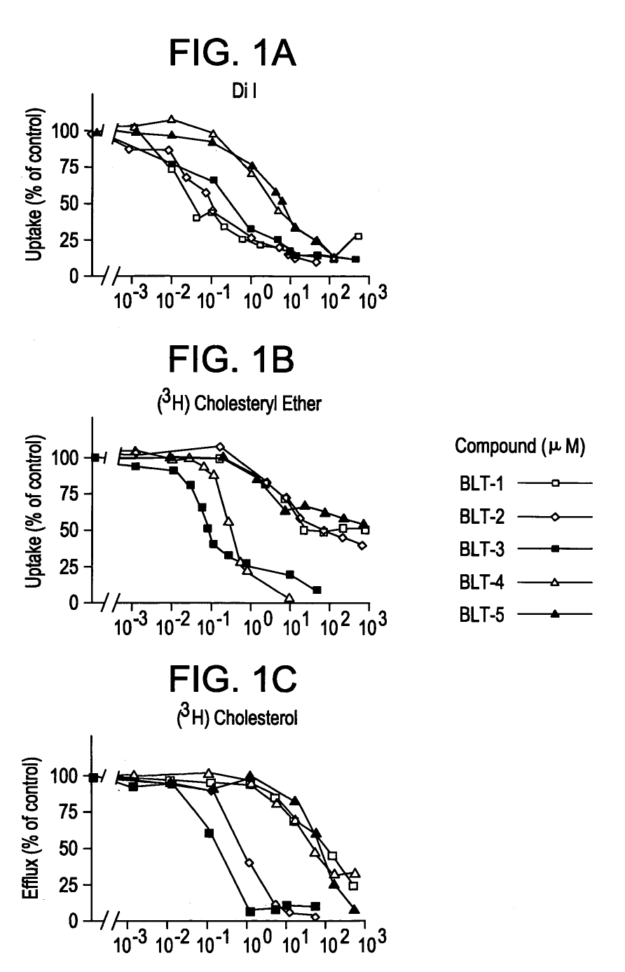

FIG. 1 shows results from a representative assay plate along with controls (no compounds, addition of excess unlabeled HDL or use of untransfected ldlA-7 cells). The figure is an example of a fluorescent read-out obtained from a single 384-well plate during the first round of the high-throughput screen. SR-BI-expressing ldlA[mSR-BI] cells were plated into 384-well plates and the effect of approximately 10 micromolar compounds on the uptake of DiI from DiI-HDL (10 μg protein/ml) was determined using a high speed fluorescence plate reader. Columns 1-20 show results (fluorescence in arbitrary units) from 16 independent wells per column (different colored symbols) from a single plate, representing a total of 320 compounds. Controls without compounds are wells either containing ldlA[mSR-BI] cells in the absence or presence of a 40-fold excess of unlabeled HDL, or containing untransfected ldlA-7 cells (very low SR-BI expression). Wells containing an inhibitory compound named BLT-1 and wells with compounds that quenched DiI-HDL fluorescence (Q) are indicated.

Compounds that quenched (‘Q’) or enhanced the intrinsic fluorescence of DiI-HDL were not examined further. Approximately 200 compounds that reproducibly blocked DiI uptake in a first round of screening were retested. These are shown in Table I.

TABLE I
Structures of SR-BI Inhibitors

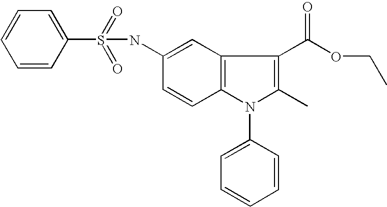

MIT 9952-1

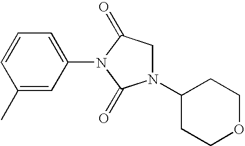

MIT 9952-2

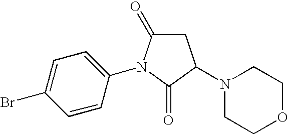

MIT 9952-3

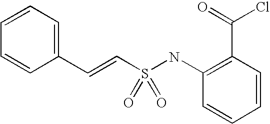

MIT 9952-4

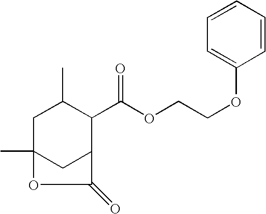

MIT 9952-5

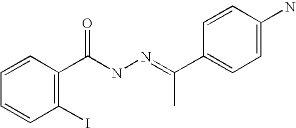

MIT 9952-6

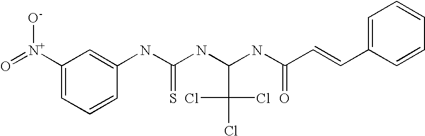

MIT 9952-7

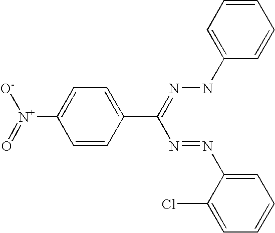

MIT 9952-8

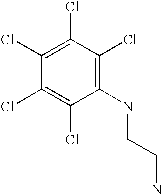

MIT 9952-9

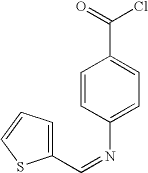

MIT 9952-10

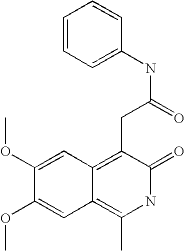

MIT 9952-11

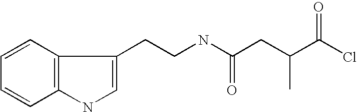

MIT 9952-12

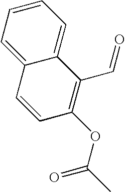

MIT 9952-13

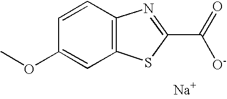

MIT 9952-14

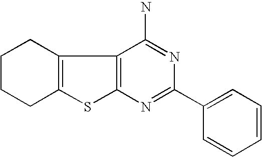

MIT 9952-15

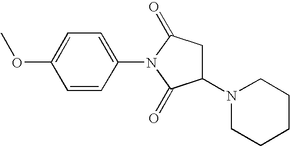

MIT 9952-16

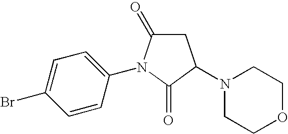

MIT 9952-17

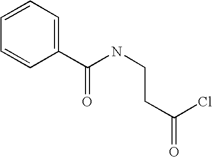

MIT 9952-18

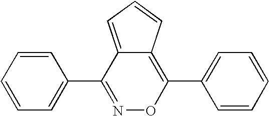

MIT 9952-19

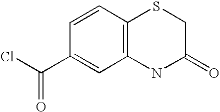

MIT 9952-20

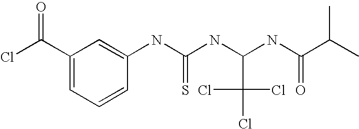

MIT 9952-21

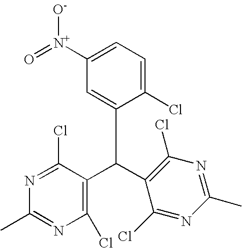

MIT 9952-22

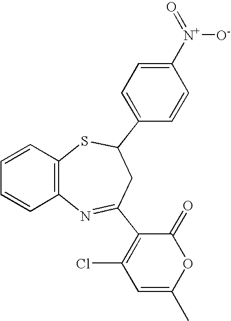

MIT 9952-23

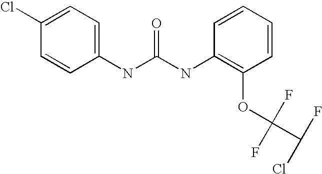

MIT 9952-24

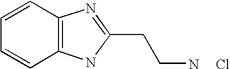

MIT 9952-25

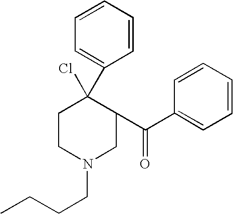

MIT 9952-26

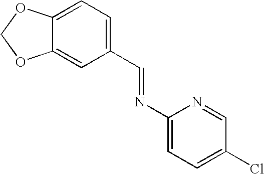

MIT 9952-27

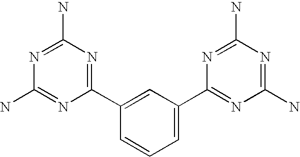

MIT 9952-28

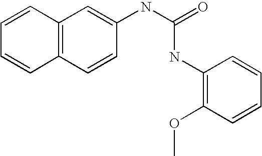

MIT 9952-29

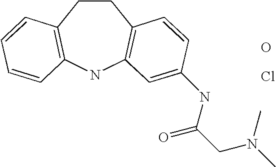

MIT 9952-30

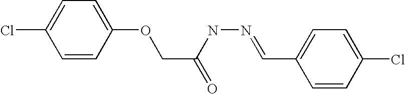

MIT 9952-31

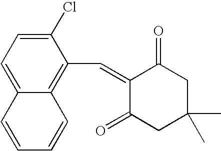

MIT 9952-32

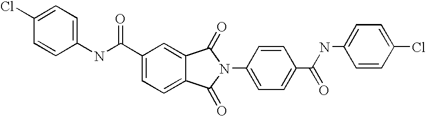

MIT 9952-33

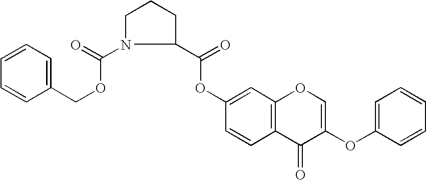

MIT 9952-34

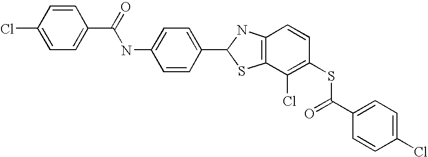

MIT 9952-35

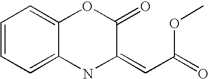

MIT 9952-36

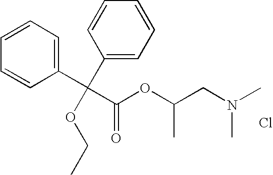

MIT 9952-37

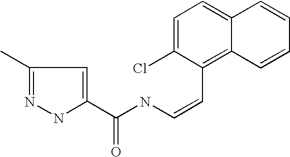

MIT 9952-38

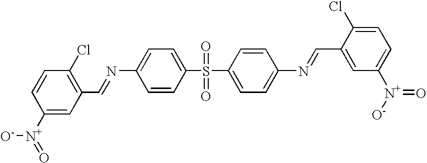

MIT 9952-39

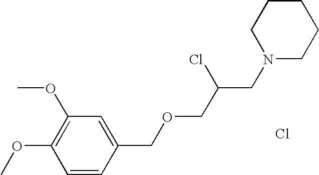

MIT 9952-40

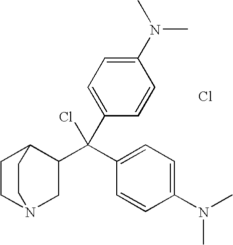

MIT 9952-41

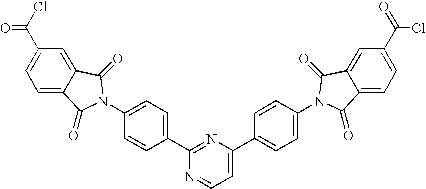

MIT 9952-42

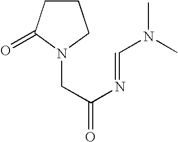

MIT 9952-43

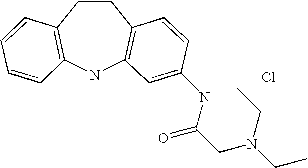

MIT 9952-44

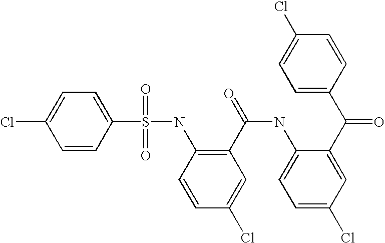

MIT 9952-45

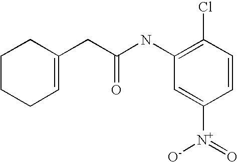

MIT 9952-46

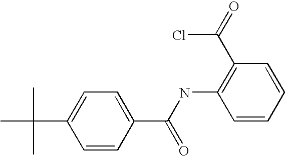

MIT 9952-47

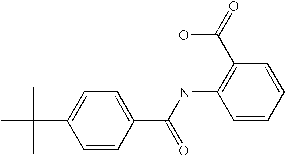

MIT 9952-48

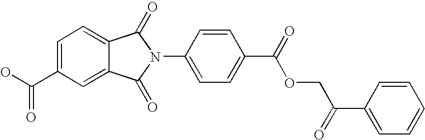

MIT 9952-49

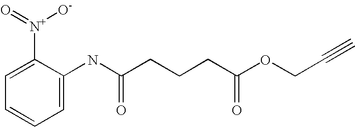

MIT 9952-50

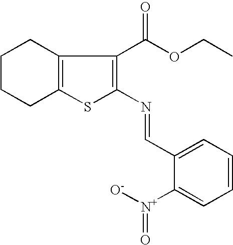

MIT 9952-51

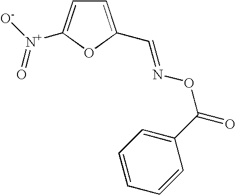

MIT 9952-52

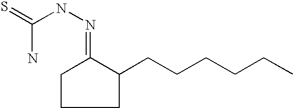

MIT 9952-53

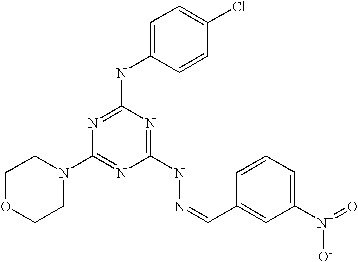

MIT 9952-54

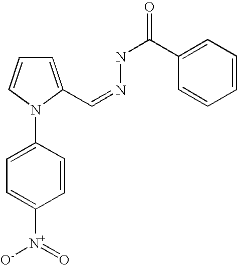

MIT 9952-55

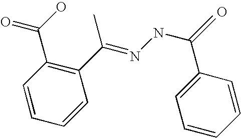

MIT 9952-56

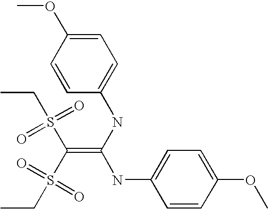

MIT 9952-57

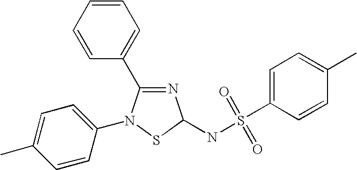

MIT 9952-58

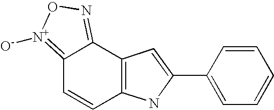

MIT 9952-59

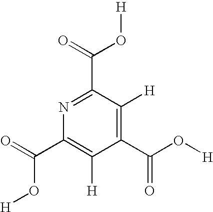

MIT 9952-60

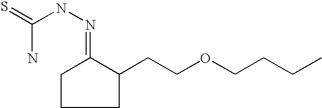

MIT 9952-61

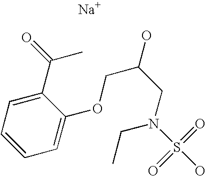

MIT 9952-62

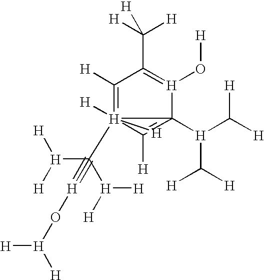

MIT 9952-63

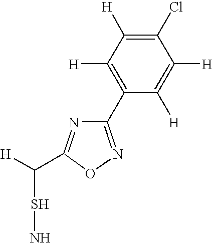

MIT 9952-64

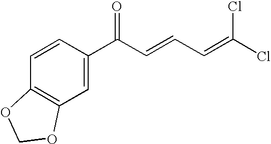

MIT 9952-65

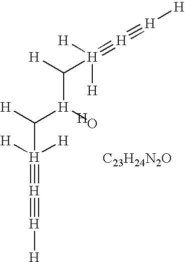

MIT 9952-66

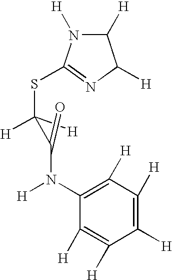

MIT 9952-67

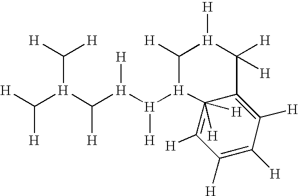

MIT 9952-68

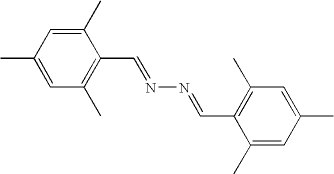

MIT 9952-69

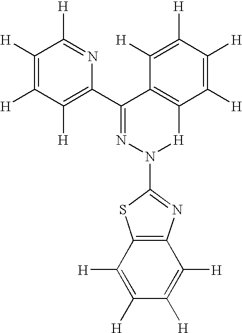

MIT 9952-70

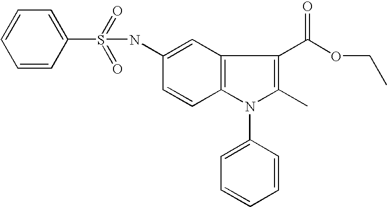

MIT 9952-71

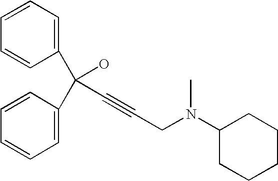

MIT 9952-72

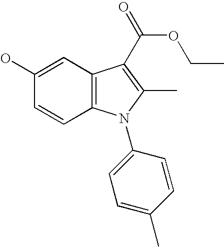

MIT 9952-73

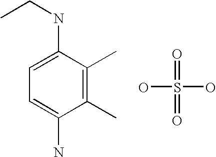

MIT 9952-74

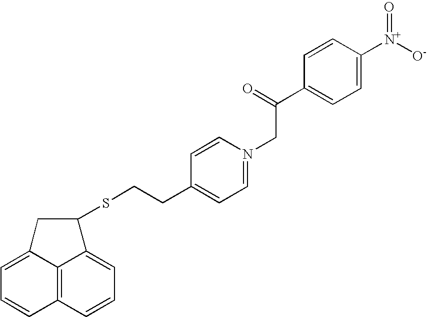

MIT 9952-75

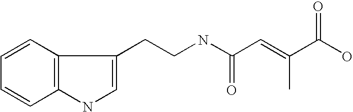

MIT 9952-76

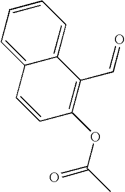

MIT 9952-77

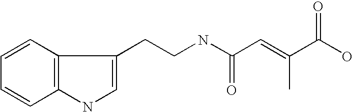

MIT 9952-78

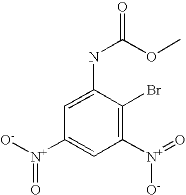

MIT 9952-79

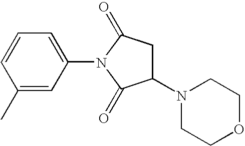

MIT 9952-80

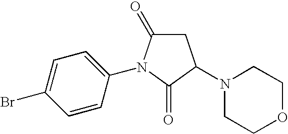

MIT 9952-81

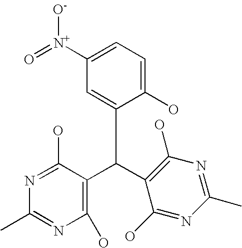

MIT 9952-82

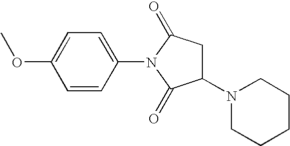

MIT 9952-83

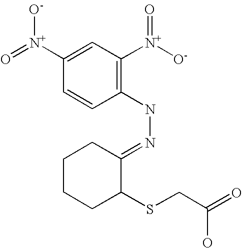

MIT 9952-84

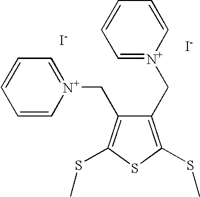

MIT 9952-85

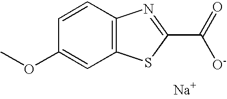

MIT 9952-86

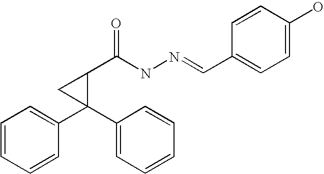

MIT 9952-87

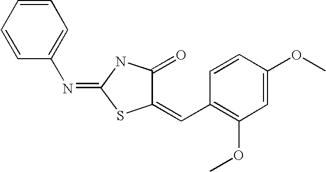

MIT 9952-88

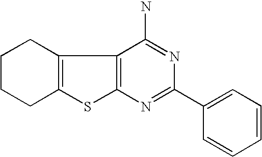

MIT 9952-89

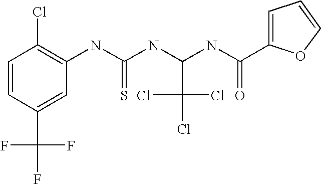

MIT 9952-90

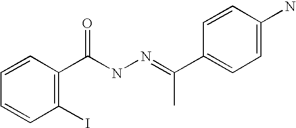

MIT 9952-91

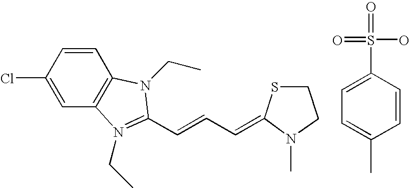

MIT 9952-92

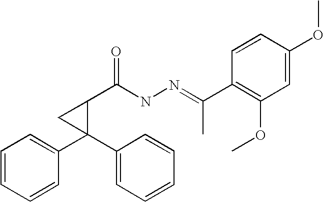

MIT 9952-93

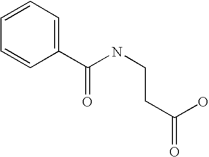

MIT 9952-94

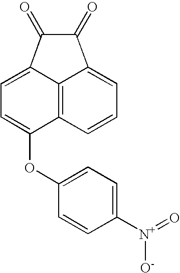

MIT 9952-95

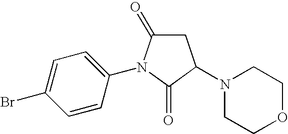

MIT 9952-96

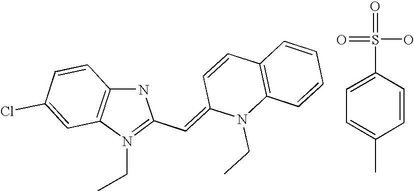

MIT 9952-97

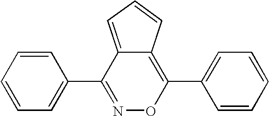

MIT 9952-98

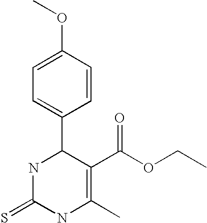

MIT 9952-99

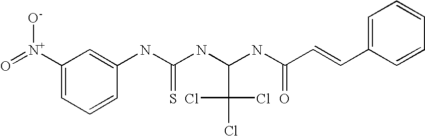

MIT 9952-100

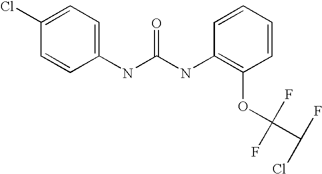

MIT 9952-101

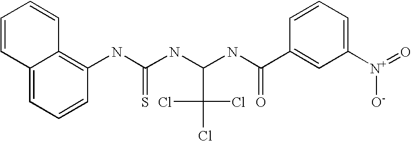

MIT 9952-102

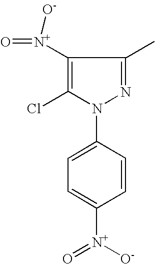

MIT 9952-103

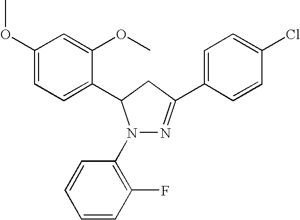

MIT 9952-104

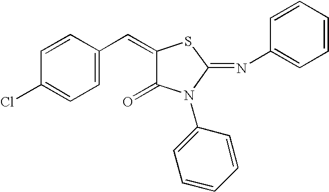

MIT 9952-105

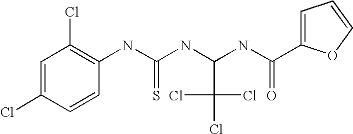

MIT 9952-106

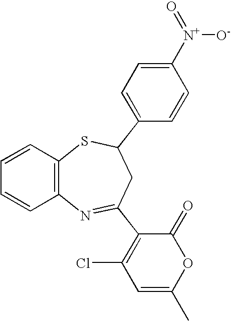

MIT 9952-107

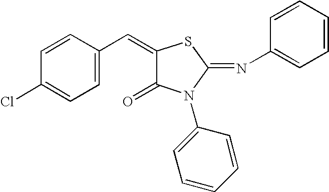

MIT 9952-108

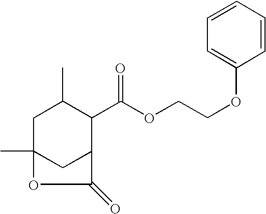

MIT 9952-109

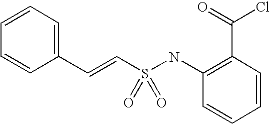

MIT 9952-110

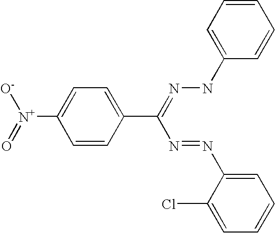

MIT 9952-111

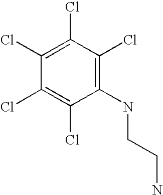

MIT 9952-112

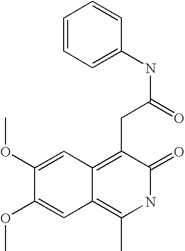

MIT 9952-113

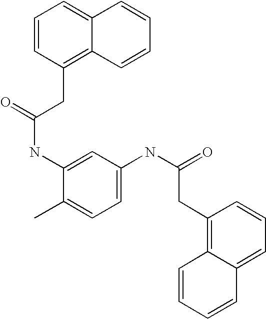

MIT 9952-114

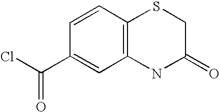

MIT 9952-115

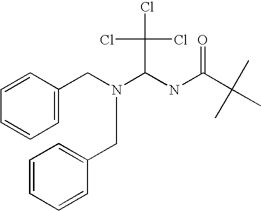

MIT 9952-116

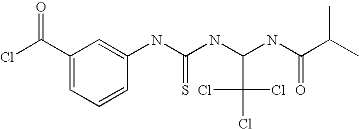

MIT 9952-117

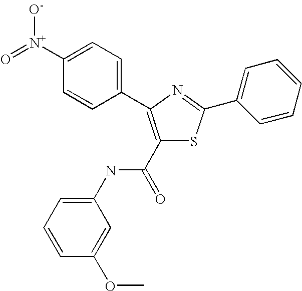

MIT 9952-118

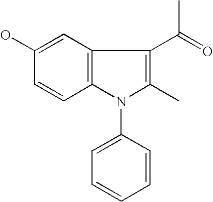

MIT 9952-119

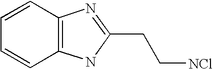

MIT 9952-120

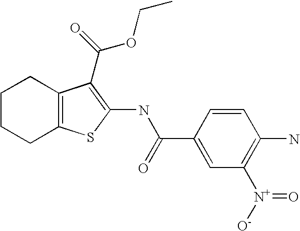

MIT 9952-121

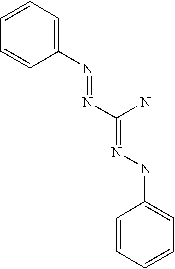

MIT 9952-122

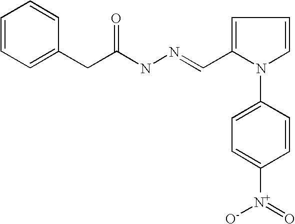

MIT 9952-123

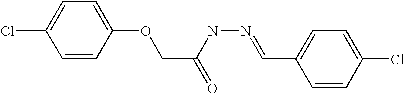

MIT 9952-124

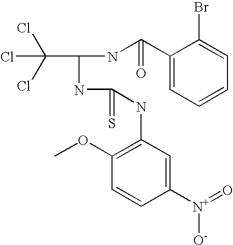

MIT 9952-125

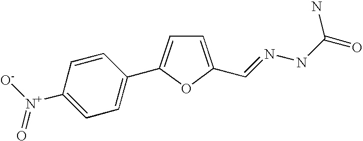

MIT 9952-126

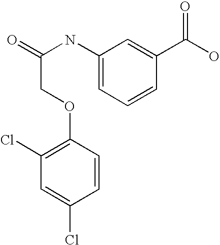

MIT 9952-127

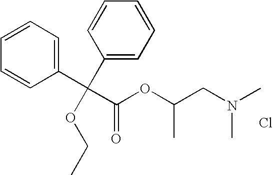

MIT 9952-128

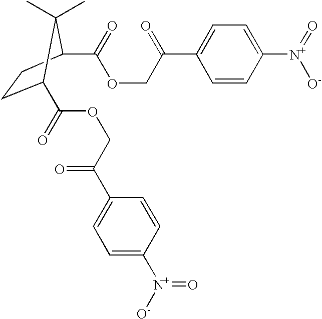

MIT 9952-129

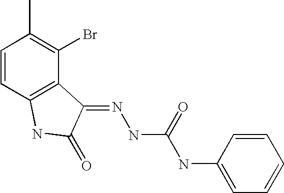

MIT 9952-130

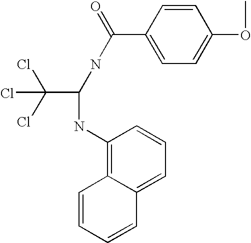

MIT 9952-131

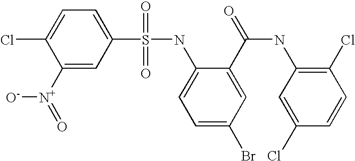

MIT 9952-132

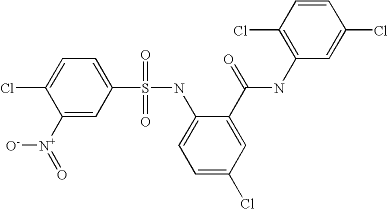

MIT 9952-133

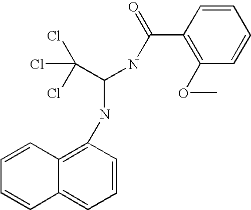

MIT 9952-134

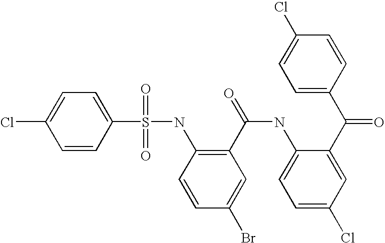

MIT 9952-135

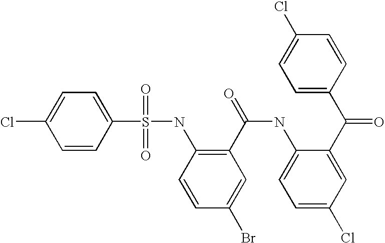

MIT 9952-136

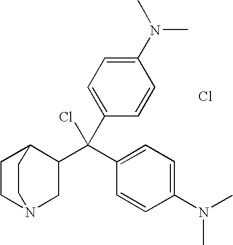

MIT 9952-137

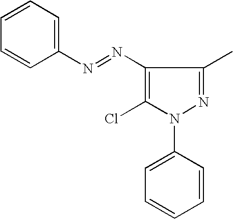

MIT 9952-138

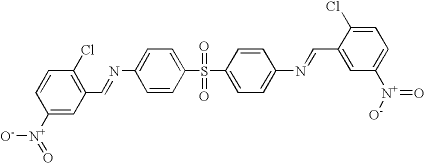

MIT 9952-139

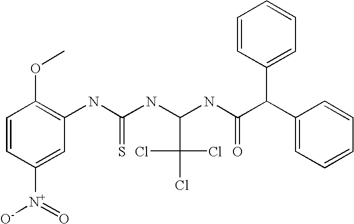

MIT 9952-140

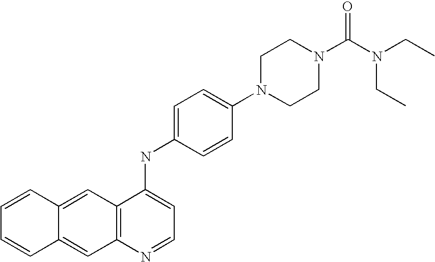

MIT 9952-141

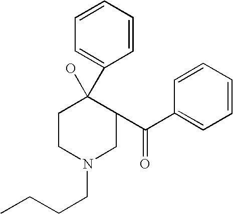

MIT 9952-142

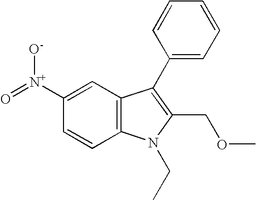

MIT 9952-143

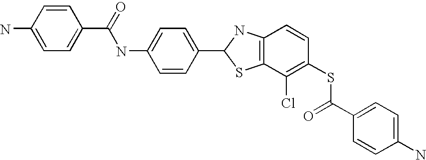

MIT 9952-144

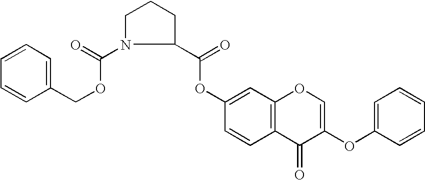

MIT 9952-145

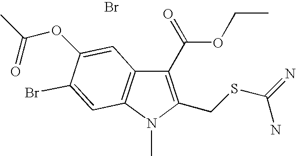

MIT 9952-146

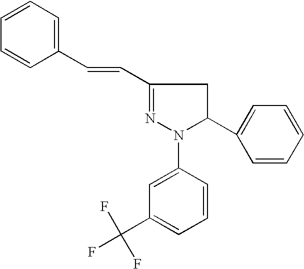

MIT 9952-147

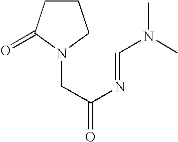

MIT 9952-148

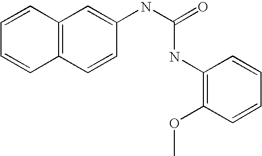

MIT 9952-149

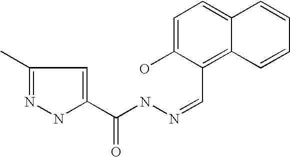

MIT 9952-150

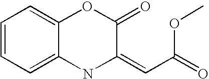

MIT 9952-151

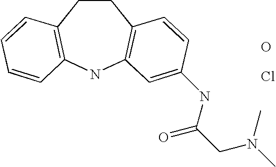

MIT 9952-152

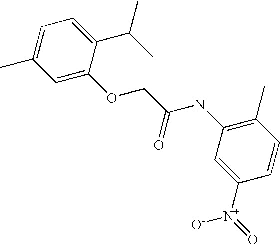

MIT 9952-153

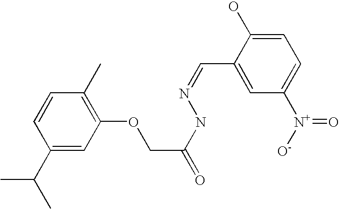

MIT 9952-154

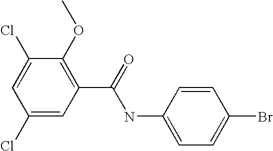

MIT 9952-155

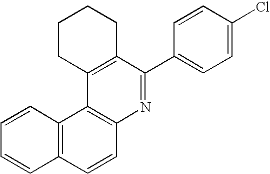

MIT 9952-156

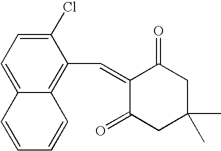

MIT 9952-157

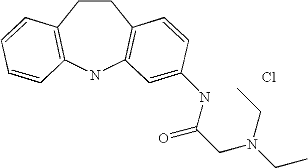

MIT 9952-158

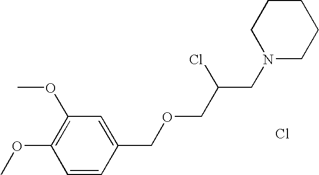

MIT 9952-159

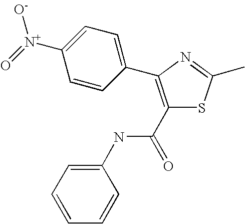

MIT 9952-160

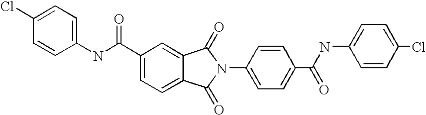

MIT 9952-161

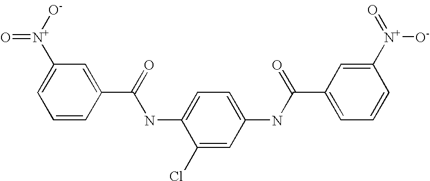

MIT 9952-162

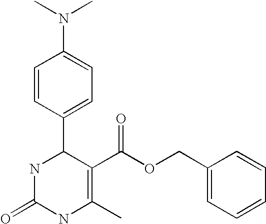

MIT 9952-163

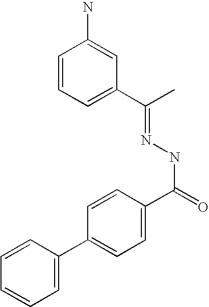

MIT 9952-164

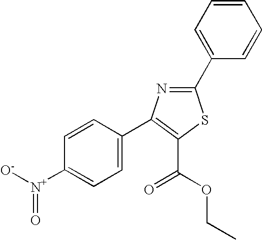

MIT 9952-165

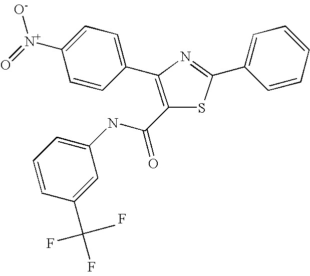

MIT 9952-166

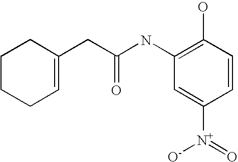

MIT 9952-167

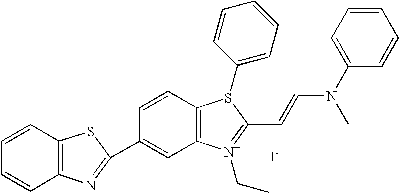

MIT 9952-168

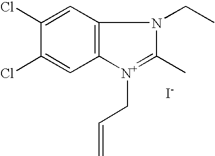

MIT 9952-169

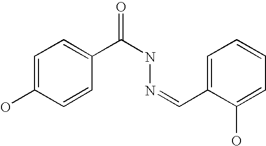

MIT 9952-170

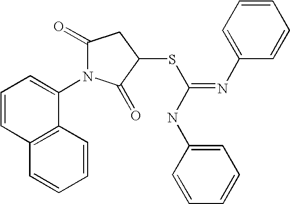

MIT 9952-171

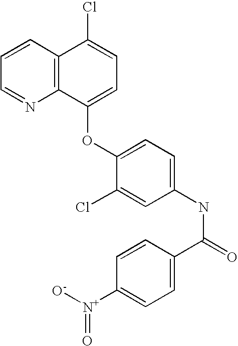

MIT 9952-172

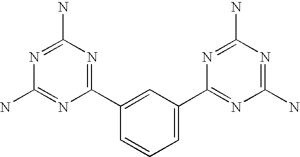

MIT 9952-173

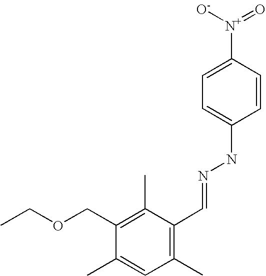

MIT 9952-174

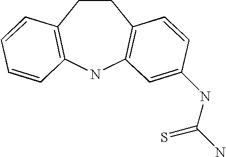

MIT 9952-175

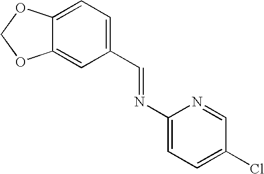

MIT 9952-176

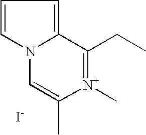

MIT 9952-177

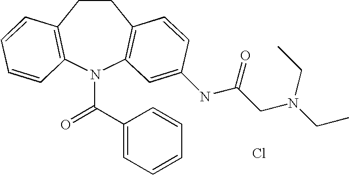

MIT 9952-178

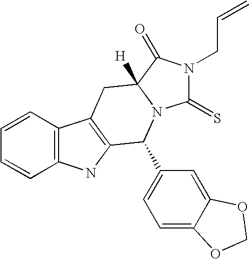

MIT 9952-179

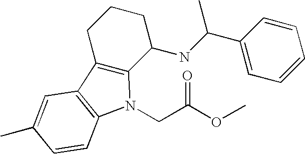

MIT 9952-180

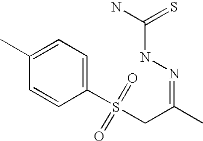

MIT 9952-181

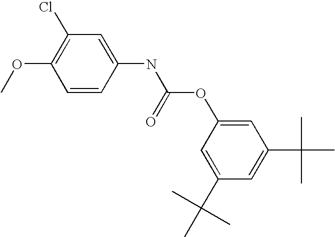

MIT 9952-182

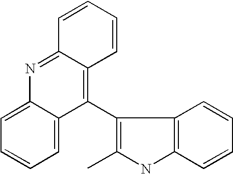

MIT 9952-183

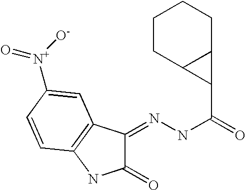

MIT 9952-184

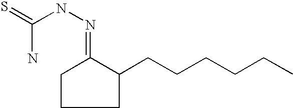

MIT 9952-185

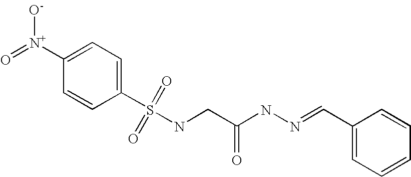

MIT 9952-186

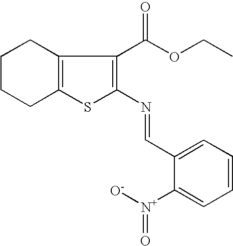

MIT 9952-187

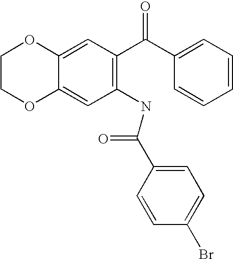

MIT 9952-188

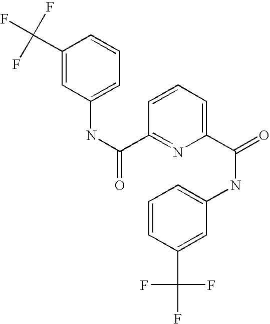

MIT 9952-189

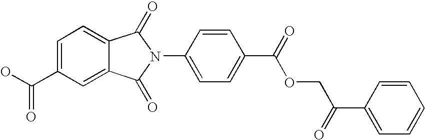

MIT 9952-190

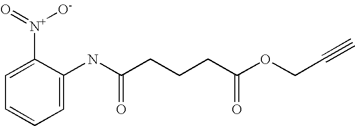

MIT 9952-191

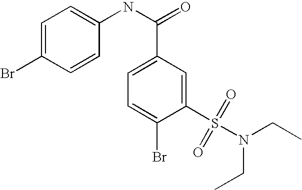

MIT 9952-192

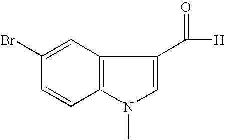

MIT 9952-193

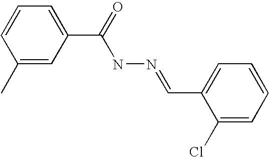

MIT 9952-194

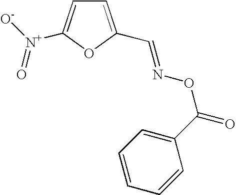

MIT 9952-195

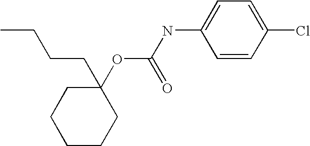

MIT 9952-196

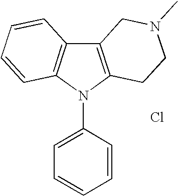

MIT 9952-197

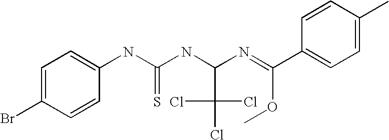

MIT 9952-198

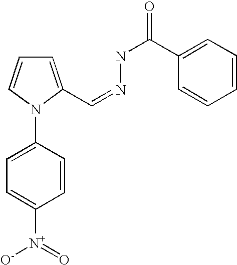

MIT 9952-199

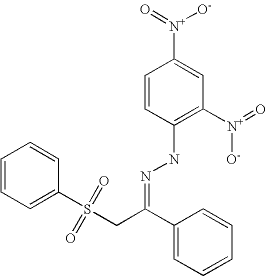

MIT 9952-200

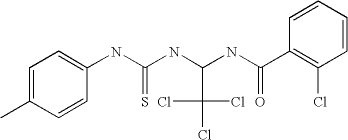

MIT 9952-201

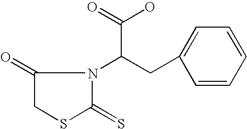

MIT 9952-202

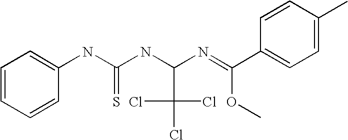

MIT 9952-203

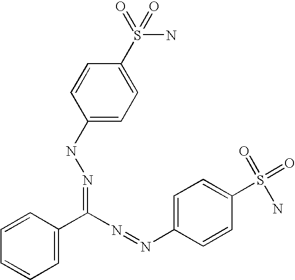

MIT 9952-204

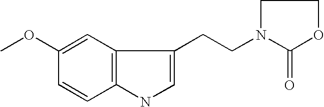

MIT 9952-205

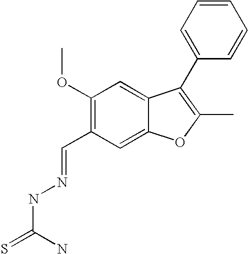

MIT 9952-206

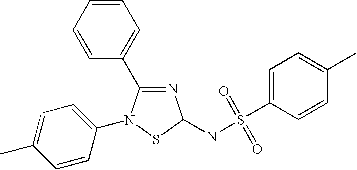

MIT 9952-207

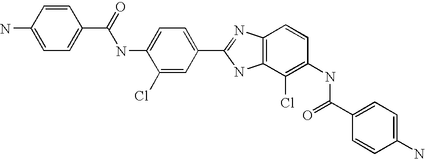

MIT 9952-208

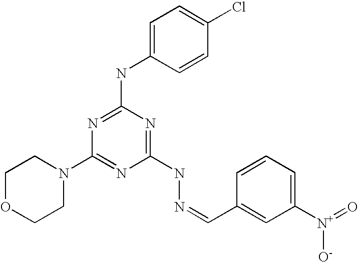

MIT 9952-209

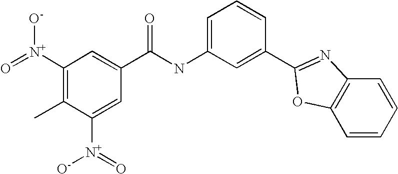

MIT 9952-210

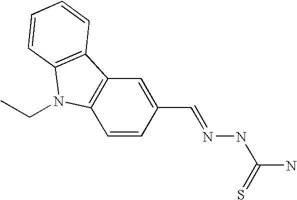

MIT 9952-211

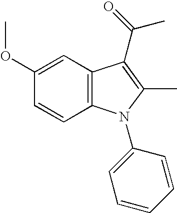

MIT 9952-212

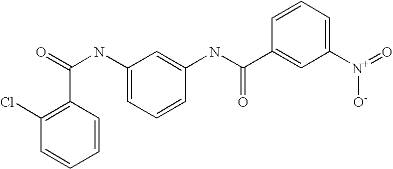

MIT 9952-213

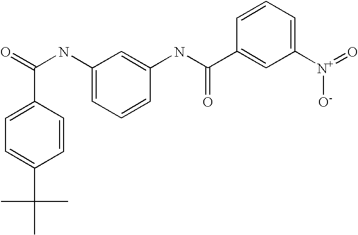

MIT 9952-214

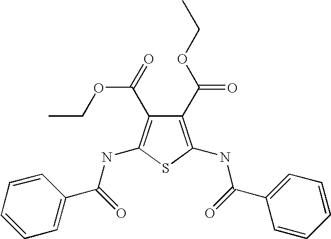

MIT 9952-215

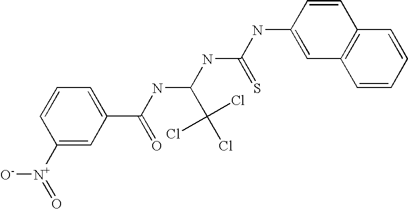

MIT 9952-216

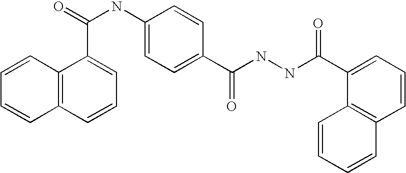

MIT 9952-217

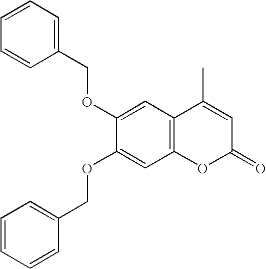

MIT 9952-218

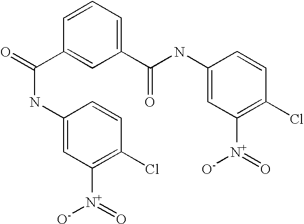

MIT 9952-219

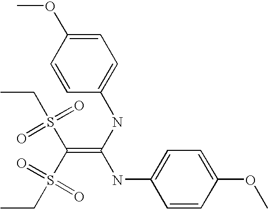

MIT 9952-220

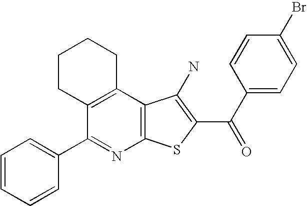

MIT 9952-221

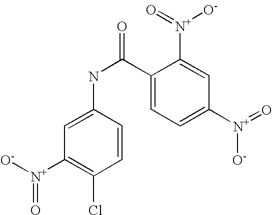

MIT 9952-222

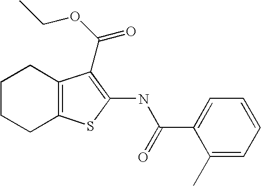

MIT 9952-223

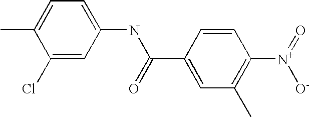

MIT 9952-224

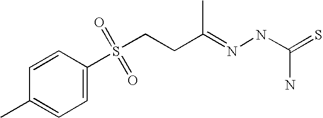

MIT 9952-225

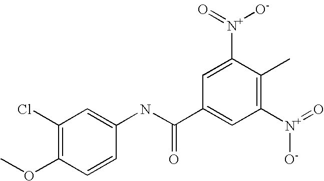

MIT 9952-226

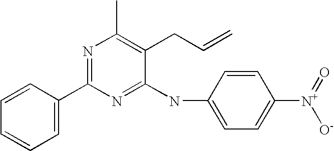

MIT 9952-227

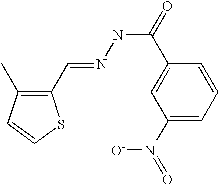

MIT 9952-228

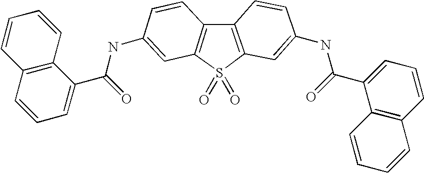

MIT 9952-229

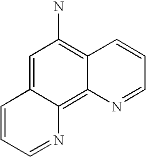

MIT 9952-230

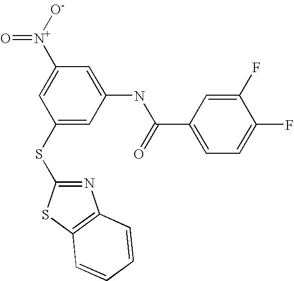

MIT 9952-231

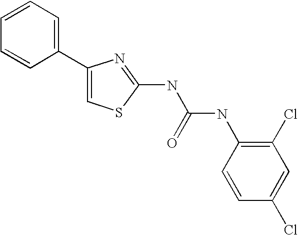

MIT 9952-232

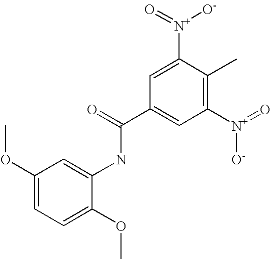

MIT 9952-233

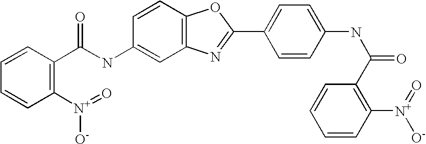

MIT 9952-234

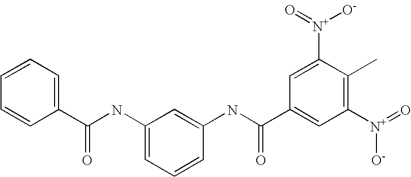

MIT 9952-235

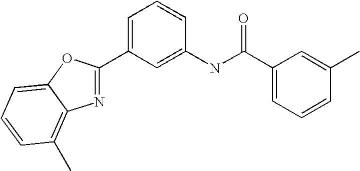

MIT 9952-236

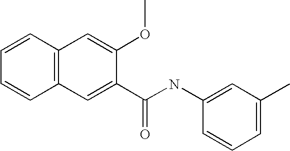

MIT 9952-237

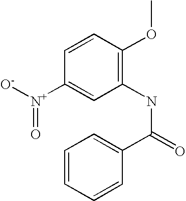

MIT 9952-238

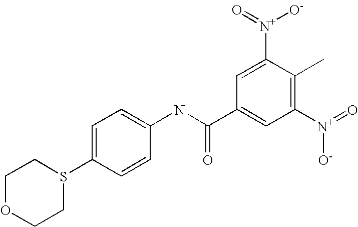

MIT 9952-239

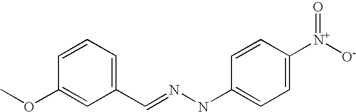

MIT 9952-240

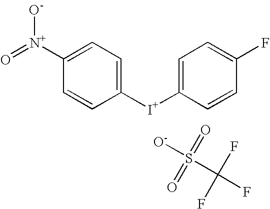

MIT 9952-241

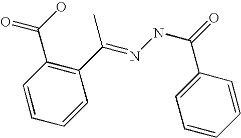

MIT 9952-242

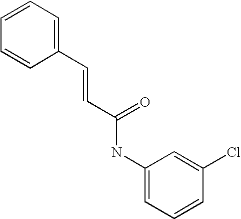

MIT 9952-243

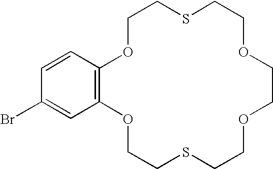

MIT 9952-244

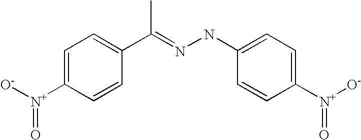

MIT 9952-245

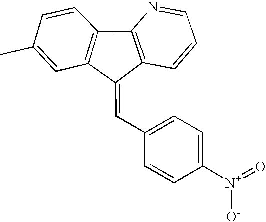

MIT 9952-246

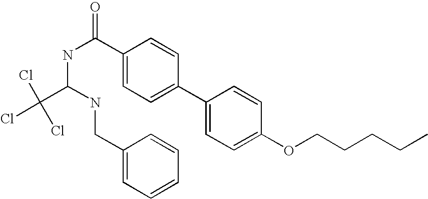

MIT 9952-247

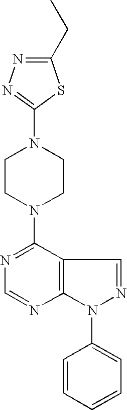

MIT 9952-248

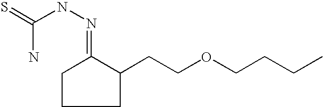

MIT 9952-249

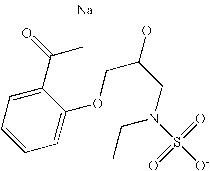

MIT 9952-250

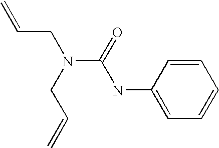

MIT 9952-251

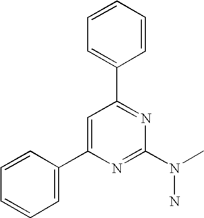

MIT 9952-252

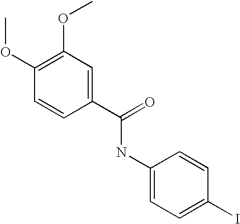

MIT 9952-253

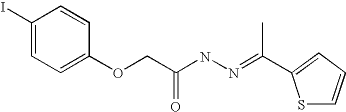

MIT 9952-254

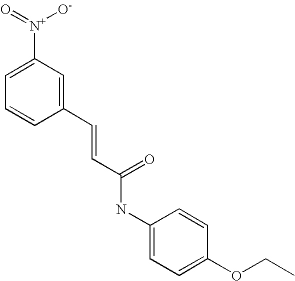

MIT 9952-255

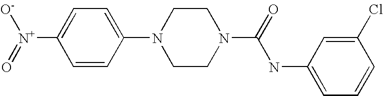

MIT 9952-256

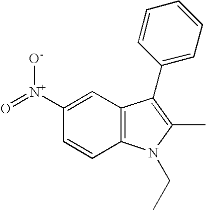

MIT 9952-257

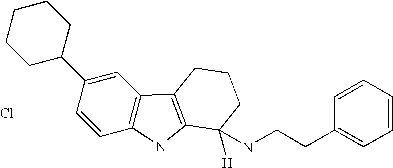

MIT 9952-258

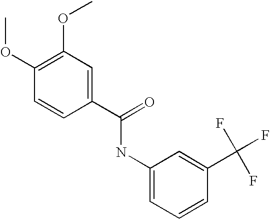

MIT 9952-259

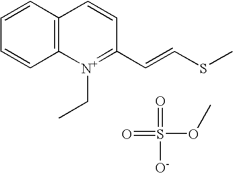

MIT 9952-260

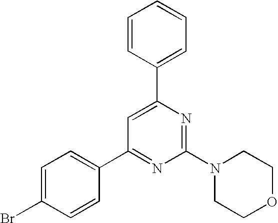

MIT 9952-261

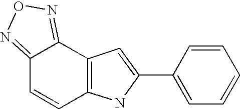

MIT 9952-262

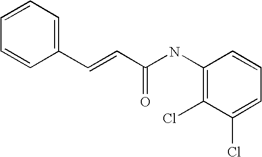

MIT 9952-263

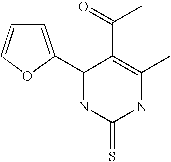

MIT 9952-264

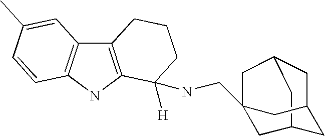

MIT 9952-265

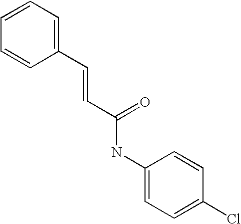

MIT 9952-266

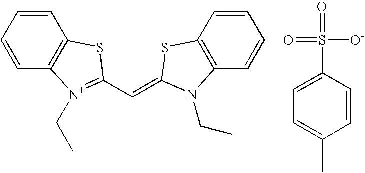

MIT 9952-267

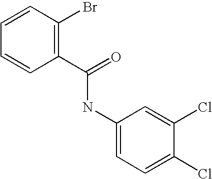

MIT 9952-268

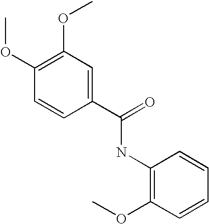

MIT 9952-269

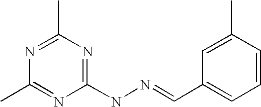

MIT 9952-270

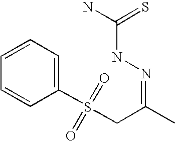

MIT 9952-271

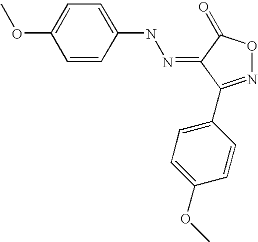

MIT 9952-272

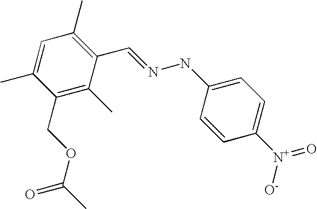

MIT 9952-273

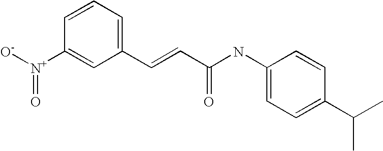

MIT 9952-274

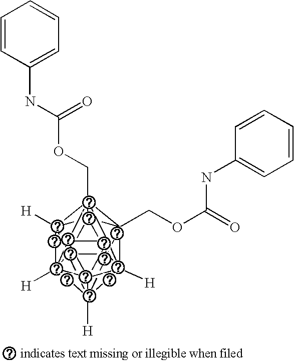

MIT 9952-275

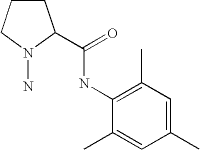

MIT 9952-276

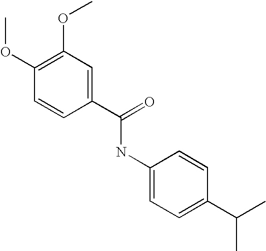

MIT 9952-277

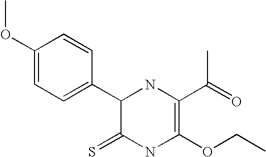

MIT 9952-278

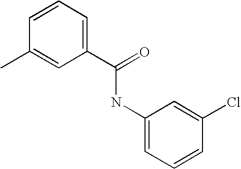

MIT 9952-279

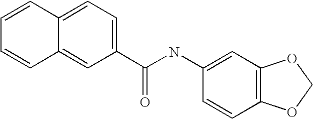

MIT 9952-280

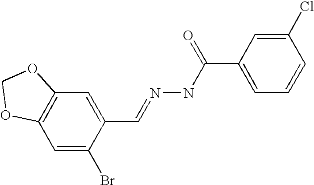

MIT 9952-281

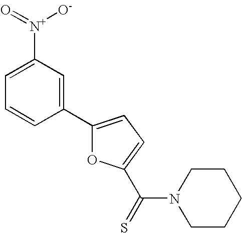

MIT 9952-282

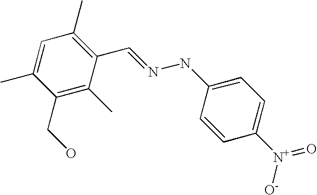

MIT 9952-283

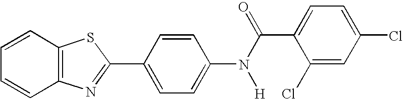

MIT 9952-284

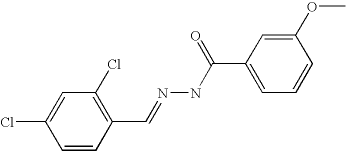

MIT 9952-285

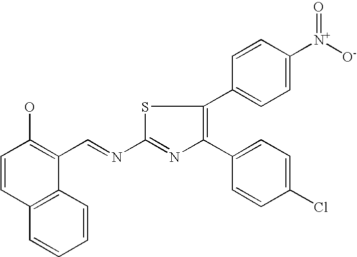

MIT 9952-286

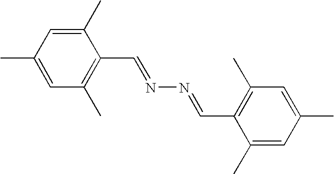

MIT 9952-287

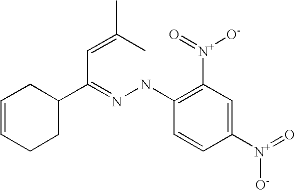

MIT 9952-288

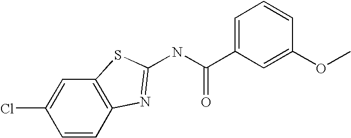

MIT 9952-289

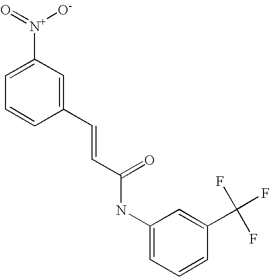

MIT 9952-290

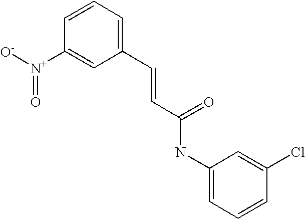

MIT 9952-291

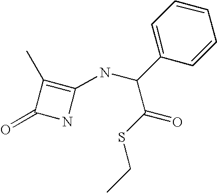

MIT 9952-292

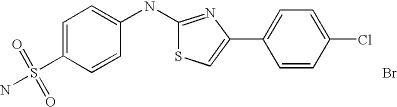

MIT 9952-293

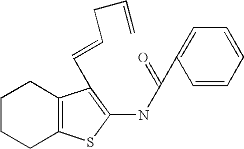

MIT 9952-294

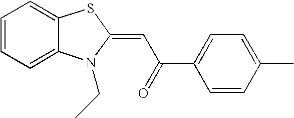

MIT 9952-295

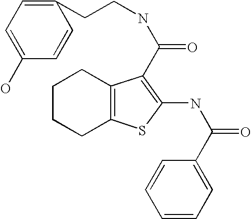

MIT 9952-296

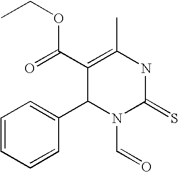

MIT 9952-297

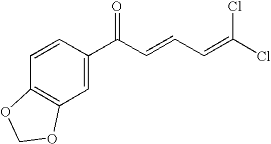

MIT 9952-298

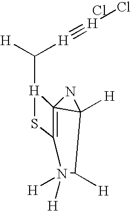

MIT 9952-299

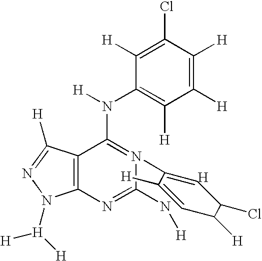

MIT 9952-300

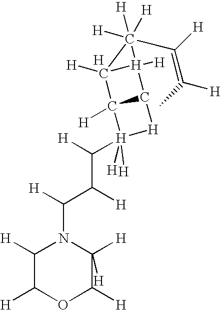

MIT 9952-301

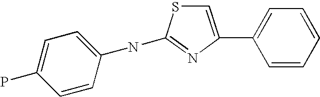

MIT 9952-302

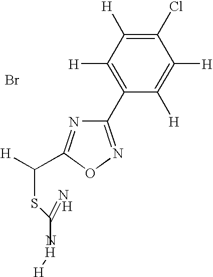

MIT 9952-303

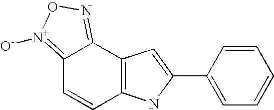

MIT 9952-304

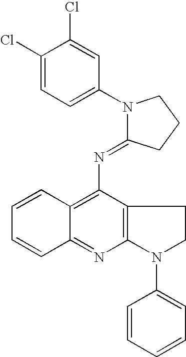

MIT 9952-305

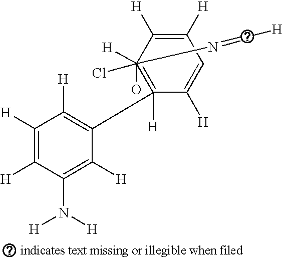

MIT 9952-306

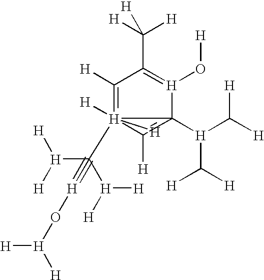

MIT 9952-307

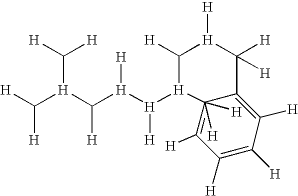

MIT 9952-308

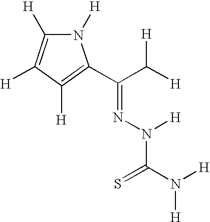

MIT 9952-309

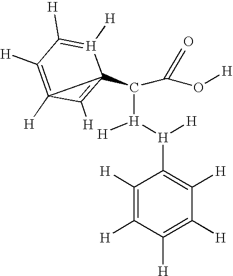

MIT 9952-310

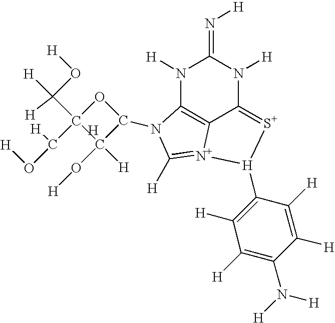

MIT 9952-311

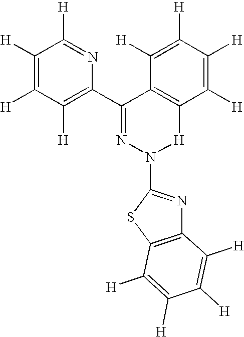

MIT 9952-312

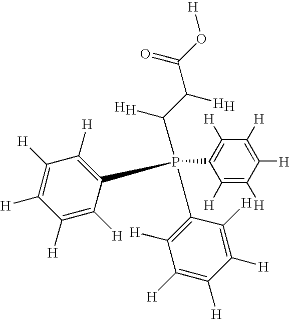

MIT 9952-313

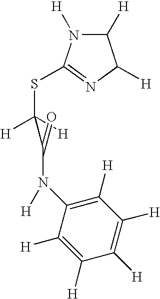

MIT 9952-314

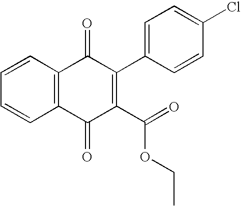

MIT 9952-315

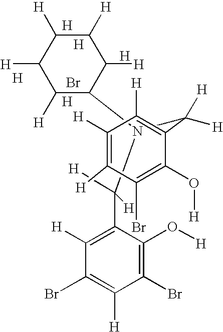

MIT 9952-316

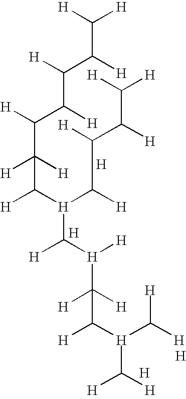

MIT 9952-317

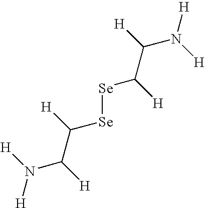

MIT 9952-318

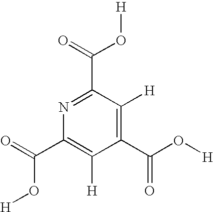

MIT 9952-319

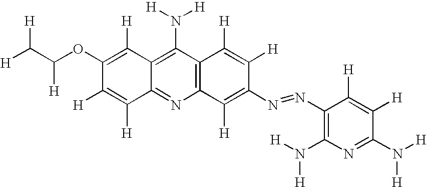

MIT 9952-320

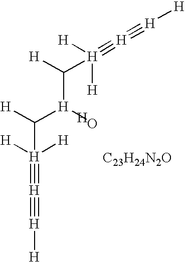

MIT 9952-321

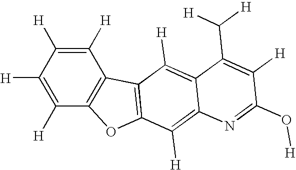

MIT 9952-322

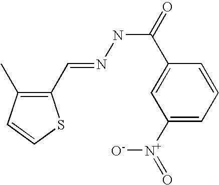

MIT 9952-323

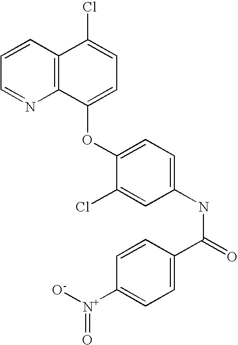

MIT 9952-324

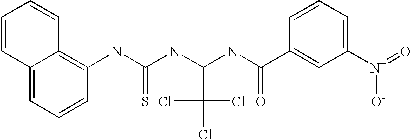

MIT 9952-325

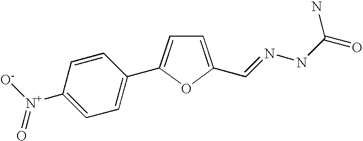

MIT 9952-326

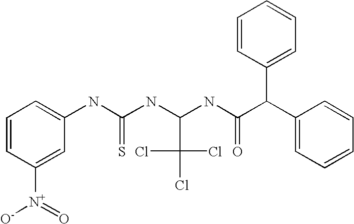

MIT 9952-327

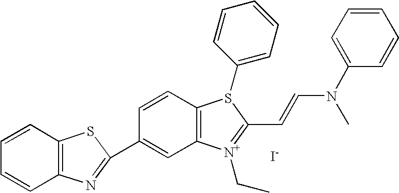

MIT 9952-328

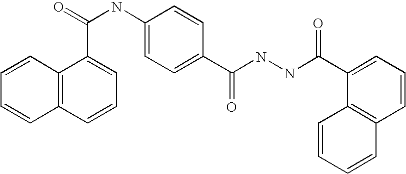

MIT 9952-329

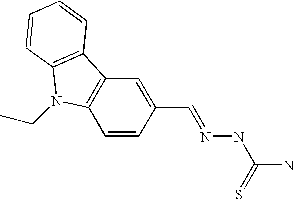

MIT 9952-330

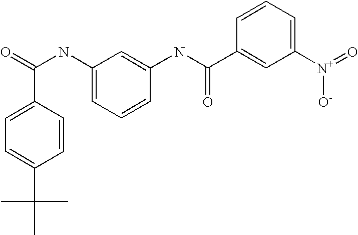

MIT 9952-331

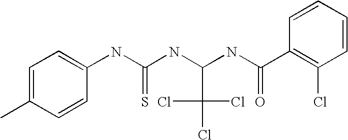

MIT 9952-332

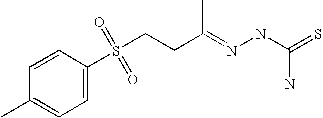

MIT 9952-333

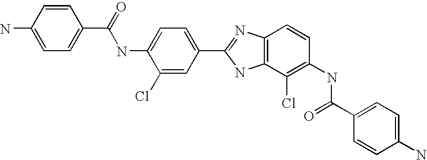

MIT 9952-334

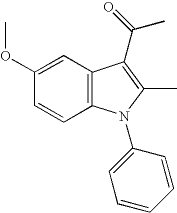

MIT 9952-335

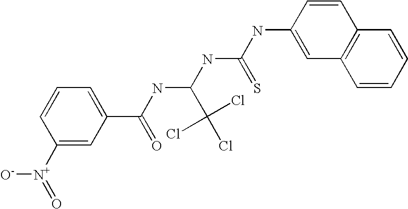

MIT 9952-336

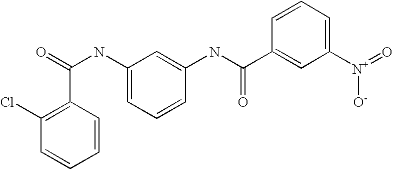

MIT 9952-337

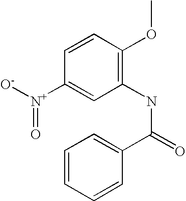

MIT 9952-338

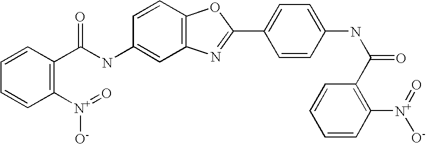

MIT 9952-339

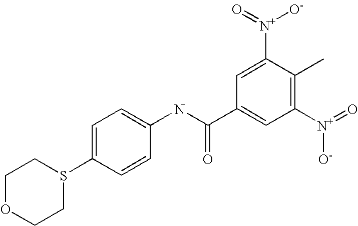

MIT 9952-340

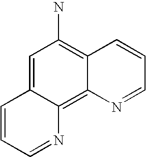

MIT 9952-341

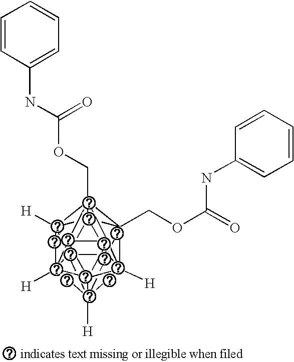

MIT 9952-342

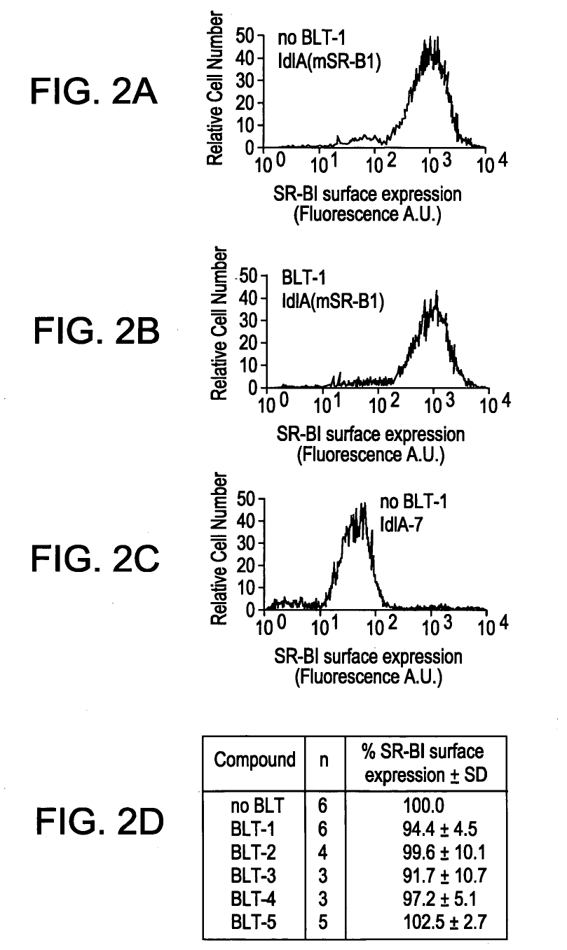

Five of the most effective compounds with IC_{DiI}50s in the micromolar or lower range (FIG. 2A) were designated BLT-1-BLT-5 and further characterized. Strikingly, the most potent of these, BLT-1 and BLT-2, inhibited in the nanomolar range and are structurally related (Table II). Inhibition of DiI uptake did not require de novo protein synthesis, because pretreatment of cells for 30 min with 100 micrograms/ml cycloheximide did not diminish their inhibitory effects. Finally, none of the BLTs substantially inhibited the low background level of uptake of DiI or [³H]CE by untransfected ldlA-7 cells expressing minimal amounts of SR-BI.

The IC_{CE}50s for inhibition of uptake of the more physiologic lipid [³H]cholesteryl ether ([³H]CE) from [³H]CE-HDL by ldlA[mSR-BI] cells were similar to those for DiI uptake (FIG. 2B and Table II). The inhibition of [³ H]CE uptake was reversible (1 hr incubation with compounds followed by 3-6 hr washout period). Moreover, the compounds also blocked the uptake of [³H]CE by YL-BS1 adrenocortical cells that express high levels of SR-BI (Rigotti, et al. (1996) J. Biol. Chem. 271, 33545-9) (Table II), indicating that the inhibitory effects by the compounds are not cell-type specific. Experiments in which the cells or the labeled HDL were pre-incubated with the compounds indicated that the cells rather than the HDL were the target of the compounds.

TABLE 2
Results of Testing for SR-BI binding.

| | ENDOVIS | | ENDOHDL | | ENDOQUENCH | |
|---|---|---|---|---|---|---|
| | Test | Test | Test | Test | Test | Test |
| Chemical ID | 1 | 2 | 1 | 2 | 1 | 2 |
| MIT 9952-1 | 0 | | 0.62 | 0.55 | 1.04 | 1.14 |
| MIT 9952-2 | 0 | | 1.34 | 1.2 | 1.1 | 1.06 |
| MIT 9952-3 | 0 | | 1.32 | 1.17 | 1.06 | 1.2 |
| MIT 9952-4 | 0 | | 1.17 | 1.33 | 1.06 | 1 |
| MIT 9952-5 | 0 | | 1.19 | 1.75 | 1.02 | 1.03 |
| MIT 9952-6 | 0 | | 0.52 | 0.54 | 0.99 | 1.03 |
| MIT 9952-7 | 0 | | 0.5 | 0.51 | 1.02 | 1.1 |
| MIT 9952-8 | | | | | | |
| MIT 9952-9 | 0 | | | | | |
| MIT 9952-10 | | | | | | |
| MIT 9952-11 | | | | | | |
| MIT 9952-12 | 0 | | 1.25 | 1.26 | 0.9 | 0.93 |
| MIT 9952-13 | 0 | | 0.55 | 0.67 | 0.94 | 0.94 |
| MIT 9952-14 | 0 | | 1.24 | 1.21 | 1.16 | 1.07 |
| MIT 9952-15 | 0 | | 0.55 | 0.61 | 0.87 | 0.81 |
| MIT 9952-16 | 0 | | 1.25 | 1.26 | 0.92 | 0.99 |
| MIT 9952-17 | 0 | | 1.32 | 1.17 | 1.06 | 1.12 |
| MIT 9952-18 | 0 | | 1.21 | 1.22 | 1.01 | 1.06 |
| MIT 9952-19 | 0 | 5 | | | | |
| MIT 9952-20 | 0 | 0 | | | | |
| MIT 9952-21 | 0 | | 1.26 | 1.58 | 0.94 | 0.94 |
| MIT 9952-22 | 0 | | 1.27 | 1.4 | 1.01 | 1 |
| MIT 9952-23 | 1 | 1 | | | | |
| MIT 9952-24 | 0 | 0 | | | | |
| MIT 9952-25 | 0 | | 1.21 | 1.69 | 0.98 | 0.98 |
| MIT 9952-26 | 0 | | 1.28 | 1.32 | 0.95 | 0.97 |
| MIT 9952-27 | 0 | | 1.36 | 1.17 | 0.9 | 0.88 |
| MIT 9952-28 | 7 | | 1.96 | 1.61 | 1.0 | 1.06 |
| MIT 9952-29 | 0 | | 0.62 | 0.6 | 0.94 | 0.99 |
| MIT 9952-30 | 0 | | 0.51 | 0.43 | 0.91 | 0.88 |
| MIT 9952-31 | 0 | | 1.33 | 1.17 | 1.01 | 1.07 |
| MIT 9952-32 | 0 | | 1.26 | 1.21 | 0.9 | 1.0 |
| MIT 9952-33 | 0 | | 1.37 | 1.5 | 1.04 | 1.0 |
| MIT 9952-34 | 0 | | 1.28 | 1.34 | 0.94 | 1.09 |
| MIT 9952-35 | 0 | | 0.56 | 0.56 | 0.99 | 0.93 |
| MIT 9952-36 | 0 | | 1.22 | 1.36 | 1.02 | 1.97 |
| MIT 9952-37 | 0 | | 1.23 | 1.36 | 1.03 | 1.13 |
| MIT 9952-38 | 0 | | 0.34 | 0.52 | 0.17 | 0.12 |
| MIT 9952-39 | 0 | | 1.22 | 1.39 | 1.08 | 1.05 |
| MIT 9952-40 | 0 | | 1.28 | 1.23 | 1.01 | 1.97 |
| MIT 9952-41 | 0 | | 1.32 | 1.25 | 1.06 | 0.92 |
| MIT 9952-42 | 0 | | 1.27 | 1.21 | 0.97 | 0.99 |
| MIT 9952-43 | 0 | | 1.44 | 1.32 | 1.09 | 1.08 |
| MIT 9952-44 | 0 | | 0.42 | 0.39 | 0.86 | 0.95 |
| MIT 9952-45 | 0 | | 0.44 | 0.46 | 1.35 | 1.27 |
| MIT 9952-46 | 0 | | 1.32 | 1.18 | 0.99 | 0.98 |
| MIT 9952-47 | 0 | | 1.18 | 1.37 | 1.14 | 0.98 |
| MIT 9952-48 | 0 | | 0.68 | 0.49 | 1.13 | 1.18 |
| MIT 9952-49 | 0 | | 1.47 | 1.35 | 0.95 | 0.91 |
| MIT 9952-50 | 0 | | 1.27 | 1.98 | 1.02 | 0.98 |
| MIT 9952-51 | 0 | | 0.33 | 0.46 | 1.02 | 0.93 |
| MIT 9952-52 | 0 | | 1.22 | 1.35 | 0.92 | 0.91 |
| MIT 9952-53 | 0 | | 0.53 | 0.46 | 1.0 | 1.03 |
| MIT 9952-54 | 6 | | 0.46 | 0.59 | 0.95 | 0.9 |
| MIT 9952-55 | 0 | | 0.52 | 0.46 | 0.98 | 1.05 |
| MIT 9952-56 | 0 | | 1.26 | 1.26 | 0.91 | 0.95 |
| MIT 9952-57 | 0 | | 1.13 | 1.25 | 1.05 | 1.96 |
| MIT 9952-58 | 0 | | 1.28 | 1.19 | 1.02 | 1.11 |
| MIT 9952-59 | 0 | | 0.35 | 0.44 | 0.86 | 0.93 |
| MIT 9952-60 | 0 | | 1.13 | 1.17 | 0.89 | 1.07 |
| MIT 9952-61 | 0 | | 0.7 | 0.57 | 1.09 | 1.01 |
| MIT 9952-62 | 0 | | 1.28 | 1.24 | 0.99 | 0.9 |
| MIT 9952-63 | 0 | | 0.69 | 0.63 | 0.95 | 0.85 |
| MIT 9952-64 | 0 | | 0.58 | 0.58 | 0.98 | 0.92 |
| MIT 9952-65 | 0 | | 1.29 | 1.24 | 1.07 | 1.02 |
| MIT 9952-66 | 0 | | 1.22 | 1.11 | 1.96 | 1.03 |
| MIT 9952-67 | 0 | | 0.55 | 0.54 | 1.24 | 0.94 |
| MIT 9952-68 | 0 | | 0.57 | 0.69 | 0.84 | 0.98 |
| MIT 9952-69 | 0 | | 1.18 | 1.32 | 1.07 | 1.1 |
| MIT 9952-70 (1^{st}) | 0 | | 0.45 | 0.75 | 0.97 | 0.88 |
| MIT 9952-71 | 0 | | 0.62 | 0.55 | 1.04 | 1.14 |
| MIT 9952-72 | 0 | 0 | 1.07 | 1.12 | 1.01 | 0.93 |
| MIT 9952-73 | 0 | 0 | 0.61 | 0.59 | 1.02 | 1.04 |
| MIT 9952-74 | 0 | 0 | 0.71 | 0.69 | 0.83 | 0.88 |
| MIT 9952-75 | 0 | 0 | 0.64 | 0.71 | 1.05 | 1.95 |
| MIT 9952-76 | 0 | | 1.25 | 1.26 | 0.9 | 0.93 |
| MIT 9952-77 | 0 | | 0.55 | 0.67 | 0.94 | 0.94 |
| MIT 9952-78 | 0 | | 1.25 | 1.26 | 0.9 | 0.93 |
| MIT 9952-79 | 1 | | 0.61 | 0.63 | 1.0 | 0.97 |
| MIT 9952-80 | 0 | | 1.34 | 1.2 | 1.1 | 1.06 |
| MIT 9952-81 | 0 | | 1.32 | 1.17 | 1.06 | 1.2 |
| MIT 9952-82 | 0 | | 1.27 | 1.4 | 1.02 | 1.0 |
| MIT 9952-83 | 0 | | 1.25 | 1.26 | 0.92 | 0.99 |
| MIT 9952-84 | 0 | 0 | 0.74 | 0.66 | 0.83 | 0.81 |
| MIT 9952-85 | 0 | 0 | 0.56 | 0.68 | 1.03 | 1.12 |
| MIT 9952-86 | 0 | | 1.24 | 1.21 | 1.16 | 1.07 |
| MIT 9952-87 | 0 | 0 | 0.67 | 0.67 | 0.9 | 0.99 |
| MIT 9952-88 | 0 | 0 | 0.73 | 0.77 | 0.92 | 0.97 |
| MIT 9952-89 | 0 | | 0.55 | 0.61 | 0.87 | 0.81 |
| MIT 9952-90 | 0 | 0 | 0.62 | 0.61 | 1.14 | 1.02 |
| MIT 9952-91 | 0 | 0 | 0.52 | 0.54 | 0.99 | 1.03 |
| MIT 9952-92 | 0 | 0 | 0.7 | 0.63 | 1.11 | 1 |
| MIT 9952-93 | 0 | 0 | 0.66 | 0.71 | 0.95 | 0.91 |
| MIT 9952-94 | 0 | | 1.21 | 1.22 | 1.01 | 1.06 |
| MIT 9952-95 | 2 | 2 | 0.79 | 0.73 | 0.96 | 1.03 |
| MIT 9952-96 | 0 | | 1.32 | 1.17 | 1.06 | 1.2 |
| MIT 9952-97 | 5 | 5 | 0.74 | 0.69 | 0.94 | 0.9 |
| MIT 9952-98 | 0 | 5 | | | | |
| MIT 9952-99 | 0 | 0 | | | | |
| MIT 9952-100 | 0 | | 0.5 | 0.51 | 1.02 | 1.1 |
| MIT 9952-101 | 0 | 0 | | | | |
| MIT 9952-102 | 0 | 0 | 0.56 | 0.49 | 1.05 | 1.05 |
| MIT 9952-103 | 1 | 1 | 0.56 | 0.61 | 0.96 | 1.09 |
| MIT 9952-104 | 0 | 0 | | | | |
| MIT 9952-105 | 0 | 0 | | | | |
| MIT 9952-106 | 0 | 0 | 0.6 | 0.53 | 1.16 | 1.16 |
| MIT 9952-107 | 1 | 1 | | | | |
| MIT 9952-108 | 0 | 0 | | | | |
| MIT 9952-109 | 0 | | 1.19 | 1.75 | 1.02 | 1.03 |
| MIT 9952-110 | 0 | | 1.17 | 1.33 | 1.06 | 1.1 |
| MIT 9952-111 | | | | | | |
| MIT 9952-112 | 0 | 0 | | | | |
| MIT 9952-113 | | | | | | |
| MIT 9952-114 | 0 | 0 | | | | |
| MIT 9952-115 | | | | | | |
| (2^{nd}) | | | | | | |
| MIT 9952-116 | 0 | 0 | | | | |
| MIT 9952-117 | 0 | | 1.26 | 1.58 | 0.94 | 0.94 |
| MIT 9952-118 | 0 | | 0.51 | 0.63 | 0.91 | 1.11 |
| MIT 9952-119 | 0 | 0 | 0.62 | 0.64 | 1.06 | 1.04 |
| MIT 9952-120 | 0 | | 1.21 | 1.69 | 0.98 | 0.98 |
| MIT 9952-121 | 0 | 0 | 0.54 | 0.57 | 0.95 | 1.02 |
| MIT 9952-122 | 0 | 0 | 0.55 | 0.82 | 0.82 | 0.82 |
| MIT 9952-123 | 0 | 0 | 0.59 | 0.64 | 1.02 | 1.05 |
| MIT 9952-124 | 0 | | 1.33 | 1.17 | 1.01 | 1.17 |
| MIT 9952-125 | 0 | 0 | 0.63 | 0.66 | 1.0 | 0.99 |
| MIT 9952-126 | 0 | 0 | 0.55 | 0.53 | 0.93 | 0.98 |
| MIT 9952-127 | 0 | 0 | 0.62 | 0.6 | 0.97 | 0.92 |
| MIT 9952-128 | 0 | | 1.23 | 1.36 | 1.03 | 1.13 |
| MIT 9952-129 | 0 | 0 | 0.57 | 0.54 | 0.8 | 0.79 |
| MIT 9952-130 | 0 | 0 | 0.62 | 0.65 | 0.96 | 1.05 |
| MIT 9952-131 | 0 | 0 | 0.56 | 0.52 | 0.9 | 0.92 |
| MIT 9952-132 | 0 | 0 | 0.65 | 0.46 | 1.15 | 1.17 |
| MIT 9952-133 | 0 | 0 | 0.5 | 0.52 | 1.15 | 1.09 |
| MIT 9952-134 | 0 | 0 | 0.58 | 0.59 | 0.9 | 0.9 |
| MIT 9952-135 | 0 | 0 | 0.44 | 0.46 | 1.35 | 1.27 |
| MIT 9952-136 | 0 | 0 | 0.63 | 0.59 | 1.12 | 1.1 |
| MIT 9952-137 | 0 | 0 | 1.32 | 1.25 | 1.06 | 0.92 |
| MIT 9952-138 | 0 | 0 | 0.54 | 0.63 | 1.11 | 1.04 |
| MIT 9952-139 | 0 | | 1.22 | 1.39 | 1.08 | 1.05 |
| MIT 9952-140 | 0 | 0 | 0.52 | 0.58 | 1.44 | 1.37 |
| MIT 9952-141 | 0 | 0 | 0.63 | 0.77 | 1.0 | 0.99 |
| MIT 9952-142 | 0 | | 1.28 | 1.32 | 0.95 | 0.97 |
| MIT 9952-143 | 0 | 0 | 0.66 | 0.65 | 1.15 | 1.03 |
| MIT 9952-144 | 0 | | 0.56 | 0.56 | 0.99 | 0.93 |
| MIT 9952-145 | 0 | | 1.28 | 1.34 | 0.94 | 1.09 |
| MIT 9952-146 | 0 | 0 | 0.62 | 0.71 | 0.95 | 1.02 |
| MIT 9952-147 | 0 | 0 | 0.63 | 0.53 | 0.9 | 0.99 |
| MIT 9952-148 | 0 | | 1.44 | 1.32 | 1.09 | 1.08 |
| MIT 9952-149 | 0 | 0 | 0.62 | 0.6 | 0.94 | 0.99 |
| MIT 9952-150 | 0 | | 0.34 | 0.52 | 0.17 | 0.16 |
| MIT 9952-151(3^{rd}) | 0 | | 1.22 | 1.36 | 1.02 | 1.97 |
| MIT 9952-152 | 0 | | 0.51 | 0.43 | 0.91 | 0.88 |
| MIT 9952-153 | 0 | 0 | 0.6 | 0.57 | 0.88 | 0.91 |
| MIT 9952-154 | 0 | 0 | 0.47 | 0.45 | 0.07 | 0.08 |
| MIT 9952-155 | 0 | 0 | 0.69 | 0.47 | 1.04 | 1.17 |
| MIT 9952-156 | 0 | 0 | 0.57 | 0.62 | 1.09 | 1.03 |
| MIT 9952-157 | 0 | 0 | 1.26 | 1.21 | 0.9 | 1.0 |
| MIT 9952-158 | 0 | | 0.42 | 0.39 | 0.86 | 0.95 |
| MIT 9952-159 | 0 | | 1.28 | 1.23 | 1.01 | 0.97 |
| MIT 9952-160 | 0 | | 0.48 | 0.55 | 0.96 | 1.0 |
| MIT 9952-161 | 0 | 0 | 1.37 | 1.5 | 1.04 | 1.0 |
| MIT 9952-162 | 0 | 0 | 0.55 | 0.4 | 1.01 | 0.95 |
| MIT 9952-163 | 0 | 0 | 0.6 | 0.69 | 1.0 | 1.01 |
| MIT 9952-164 | 0 | | 0.6 | 0.61 | 0.88 | 0.89 |
| MIT 9952-165 | 0 | | 0.57 | 0.6 | 0.93 | 0.94 |
| MIT 9952-166 | 0 | | 0.56 | 0.67 | 0.95 | 0.99 |
| MIT 9952-167 | 0 | | 1.32 | 1.18 | 0.99 | 0.98 |
| MIT 9952-168 | 4 | 4 | 0.5 | 0.56 | 0.93 | 1.12 |
| MIT 9952-169 | 0 | 0 | 0.54 | 0.6 | 1.0 | 1.04 |
| MIT 9952-170 | 0 | 0 | 0.54 | 0.54 | 0.12 | 0.1 |
| MIT 9952-171 | 0 | 0 | 0.58 | 0.54 | 0.96 | 1.03 |
| MIT 9952-172 | 0 | 0 | 0.55 | 0.56 | 0.92 | 0.84 |
| MIT 9952-173 | 7 | | 1.96 | 1.61 | 1.0 | 1.06 |
| MIT 9952-174 | 0 | 0 | 0.6 | 0.62 | 0.85 | 0.84 |
| MIT 9952-175 | 0 | 0 | 0.42 | 0.51 | 1.0 | 0.98 |
| MIT 9952-176 | 0 | | 1.36 | 1.17 | 0.9 | 0.88 |
| MIT 9952-177 | 0 | 0 | 0.68 | 0.49 | 1.13 | 1.18 |
| MIT 9952-178 | 0 | 0 | 0.4 | 0.38 | 0.95 | 0.86 |
| MIT 9952-179 | 0 | 0 | 0.54 | 0.54 | 1.08 | 1.04 |
| MIT 9952-180(4^{th}) | 0 | 0 | 0.43 | 0.45 | 1.14 | 1.02 |
| MIT 9952-181 | 0 | 0 | 0.6 | 0.54 | 1.07 | 0.95 |
| MIT 9952-182 | 0 | 0 | 0.71 | 0.41 | 0.95 | 1.1 |
| MIT 9952-183 | 0 | 0 | 0.59 | 0.65 | 0.94 | 1.0 |
| MIT 9952-184 | 0 | 0 | 0.6 | 0.58 | 0.93 | 0.94 |
| MIT 9952-185 | 0 | 0 | 0.53 | 0.46 | 1.0 | 1.03 |
| MIT 9952-186 | 0 | 0 | 0.5 | 0.5 | 1.07 | 1.05 |
| MIT 9952-187 | 0 | | 0.33 | 0.46 | 1.02 | 0.93 |
| MIT 9952-188 | 0 | 0 | 0.61 | 0.58 | 0.94 | 1.08 |
| MIT 9952-189 | 0 | 0 | 0.56 | 0.58 | 1.09 | 1.0 |
| MIT 9952-190 | 0 | | 1.47 | 1.35 | 0.95 | 0.91 |
| MIT 9952-191 | 0 | | 1.27 | 1.98 | 1.02 | 0.98 |
| MIT 9952-192 | 0 | 0 | 0.57 | 0.52 | 1.1 | 1.09 |
| MIT 9952-193 | 0 | 0 | 0.66 | 0.69 | 0.92 | 1.0 |
| MIT 9952-194 | 0 | 0 | 0.76 | 0.46 | 0.97 | 1.02 |
| MIT 9952-195 | 0 | | 1.22 | 1.35 | 0.92 | 0.91 |
| MIT 9952-196 | 0 | 0 | 0.63 | 0.6 | 1.09 | 1.07 |
| MIT 9952-197 | 0 | 0 | 0.58 | 0.71 | 0.95 | 0.96 |
| MIT 9952-198 | 0 | 0 | 0.67 | 0.64 | 1.07 | 1.11 |
| MIT 9952-199 | 0 | 0 | 0.52 | 0.46 | 0.98 | 1.05 |
| MIT 9952-200 | 0 | 0 | 0.73 | 0.8 | 1.02 | 0.96 |
| MIT 9952-201 | 0 | 0 | 0.69 | 0.67 | 1.26 | 1.25 |
| MIT 9952-202 | 0 | 0 | 1.23 | 1.11 | 0.98 | 1.03 |
| MIT 9952-203 | 0 | 0 | 0.73 | 0.7 | 0.97 | 1.0 |
| MIT 9952-204 | 0 | 0 | 0.55 | 0.62 | 0.78 | 1.07 |
| MIT 9952-205 | 0 | 0 | 1.08 | 1.0 | 0.93 | 1.03 |
| MIT 9952-206 | 0 | 0 | 0.56 | 0.52 | 1.05 | 1.1 |
| MIT 9952-207 | 0 | | 1.28 | 1.19 | 1.02 | 1.11 |
| MIT 9952-208 | 0 | 0 | 0.57 | 0.55 | 0.95 | 0.98 |
| MIT 9952-209 | 6 | | 0.46 | 0.59 | 0.95 | 0.9 |
| (5^{th}) | | | | | | |
| MIT 9952-210 | 0 | 0 | 0.59 | 0.56 | 0.88 | 0.91 |
| MIT 9952-211 | 0 | 0 | 0.59 | 0.56 | 1.02 | 1.07 |
| MIT 9952-212 | 0 | 0 | 0.57 | 0.49 | 1.0 | 0.95 |
| MIT 9952-213 | 0 | 0 | 0.66 | 0.57 | 0.92 | 0.96 |
| MIT 9952-214 | 0 | 0 | 0.63 | 0.35 | 1.05 | 1.0 |
| MIT 9952-215 | 0 | 0 | 0.57 | 0.53 | 1.03 | 1.04 |
| MIT 9952-216 | 0 | 0 | 0.54 | 0.58 | 1.1 | 1.14 |
| MIT 9952-217 | 0 | 0 | 0.57 | 0.53 | 1.0 | 0.98 |
| MIT 9952-218 | 0 | 0 | 0.64 | 0.33 | 1.06 | 1.0 |
| MIT 9952-219 | 0 | 0 | 0.55 | 0.55 | 0.95 | 0.98 |
| MIT 9952-220 | 0 | 0 | 1.13 | 1.25 | 1.05 | 0.96 |
| MIT 9952-221 | 0 | 0 | 0.62 | 0.59 | 1.01 | 0.91 |
| MIT 9952-222 | 4 | 4 | 0.58 | 0.6 | 1.07 | 0.9 |
| MIT 9952-223 | 0 | 0 | 0.64 | 0.57 | 1.06 | 1.05 |
| MIT 9952-224 | 0 | 0 | 0.6 | 0.5 | 0.99 | 0.97 |
| MIT 9952-225 | 0 | 0 | 0.56 | 0.59 | 1.05 | 1.03 |
| MIT 9952-226 | 0 | 0 | 0.5 | 0.56 | 0.95 | 1.0 |
| MIT 9952-227 | 0 | 0 | 0.58 | 0.53 | 0.96 | 1.0 |
| MIT 9952-228 | 0 | 0 | 0.46 | 0.63 | 0.93 | 0.94 |
| MIT 9952-229 | 0 | 0 | 0.58 | 0.58 | 1.22 | 1.31 |
| MIT 9952-230 | 2 | 2 | 0.61 | 0.51 | 0.99 | 1.01 |
| MIT 9952-231 | 0 | 0 | 0.46 | 0.54 | 0.99 | 0.96 |
| MIT 9952-232 | 0 | 0 | 0.61 | 0.56 | 0.99 | 1.02 |
| MIT 9952-233 | 0 | 0 | 0.59 | 0.33 | 1.0 | 0.94 |
| MIT 9952-234 | 0 | 0 | 0.58 | 0.54 | 0.94 | 0.93 |
| MIT 9952-235 | 0 | 0 | 0.62 | 0.33 | 0.91 | 1.06 |
| MIT 9952-236 | 0 | 0 | 0.57 | 0.38 | 0.97 | 1.23 |
| MIT 9952-237 | 0 | 0 | 0.53 | 0.39 | 0.91 | 0.83 |
| MIT 9952-238 | 0 | 0 | 0.61 | 0.6 | 1.01 | 1.13 |
| MIT 9952-239 | 0 | 0 | 0.48 | 0.4 | 0.9 | 0.96 |
| (6^{th}) | | | | | | |
| MIT 9952-240 | 0 | 0 | 0.64 | 0.71 | 0.97 | 1.07 |
| MIT 9952-241 | 1 | 1 | 0.48 | 0.52 | 0.92 | 0.93 |
| MIT 9952-242 | 0 | | 1.26 | 1.26 | 0.91 | 0.95 |
| MIT 9952-243 | 0 | 0 | 0.42 | 0.6 | 1.05 | 1.09 |
| MIT 9952-244 | 0 | 0 | 0.56 | 0.54 | 1.02 | 1.07 |
| MIT 9952-245 | 0 | 0 | 0.54 | 0.64 | 1.03 | 1.02 |
| MIT 9952-246 | 0 | 0 | 0.56 | 0.52 | 0.99 | 0.98 |
| MIT 9952-247 | 0 | 0 | 0.63 | 0.64 | 1.05 | 1.03 |
| MIT 9952-248 | 0 | 0 | 0.68 | 0.66 | 0.98 | 0.91 |
| MIT 9952-249 | 0 | 0 | 0.7 | 0.57 | 1.09 | 1.01 |
| MIT 9952-250 | 0 | 0 | 1.28 | 1.24 | 0.99 | 0.9 |
| MIT 9952-251 | 0 | 0 | 0.52 | 0.57 | 1.06 | 1.06 |
| MIT 9952-252 | 1 | 1 | 0.58 | 0.39 | 0.98 | 0.9 |
| MIT 9952-253 | 0 | 0 | 0.59 | 0.65 | 1.03 | 1.06 |
| MIT 9952-254 | | | 0.69 | 1.01 | 0.91 | 1.05 |
| MIT 9952-255 | 0 | 0 | 0.61 | 0.6 | 1.01 | 0.94 |
| MIT 9952-256 | 0 | 0 | 0.65 | 0.92 | 0.92 | 0.97 |
| MIT 9952-257 | 0 | 0 | 0.66 | 0.61 | 1.0 | 1.0 |
| MIT 9952-258 | 0 | 0 | 0.51 | 1.0 | 0.88 | 0.82 |
| MIT 9952-259 | 0 | 0 | 0.59 | 0.55 | 0.96 | 0.94 |
| MIT 9952-260 | 0 | 0 | 0.56 | 0.58 | 1.06 | 1.04 |
| MIT 9952-261 | 0 | 0 | 0.62 | 0.66 | 1.05 | 1.05 |
| MIT 9952-262 | 0 | 0 | 0.53 | 0.45 | 0.98 | 1.01 |
| MIT 9952-263 | 0 | 0 | 0.66 | 0.65 | 1.04 | 0.98 |
| MIT 9952-264 | 0 | 0 | 0.45 | 0.56 | 1.1 | 1.11 |
| MIT 9952-265 | 0 | 0 | 0.26 | 0.89 | 0.8 | 0.87 |
| MIT 9952-266 | 0 | 0 | 0.71 | 0.68 | 1.08 | 1.01 |
| MIT 9952-267 | 0 | 0 | 0.57 | 1.11 | 0.96 | 1.07 |
| MIT 9952-268 | 0 | 0 | 0.59 | 0.65 | 0.98 | 1.04 |
| (7^{th}) | | | | | | |
| MIT 9952-269 | 0 | 0 | 0.74 | 0.66 | 0.99 | 1.05 |
| MIT 9952-270 | 0 | 0 | 0.66 | 0.66 | 0.95 | 0.96 |
| MIT 9952-271 | 0 | 0 | 0.59 | 0.54 | 0.94 | 0.89 |
| MIT 9952-272 | 0 | 0 | 0.61 | 0.51 | 0.91 | 0.92 |
| MIT 9952-273 | 0 | 0 | 0.51 | 0.48 | 0.79 | 0.73 |
| MIT 9952-274 | 0 | 0 | 0.65 | 0.6 | 0.93 | 0.93 |
| MIT 9952-275 | 0 | 0 | 0.43 | 0.44 | 0.92 | 0.97 |
| MIT 9952-276 | 0 | 0 | 0.73 | 0.68 | 1.03 | 1.0 |
| MIT 9952-277 | 0 | 0 | 0.66 | 0.65 | 1.0 | 1.0 |
| MIT 9952-278 | 0 | 0 | 0.71 | 0.67 | 1.09 | 0.98 |
| MIT 9952-279 | 0 | 0 | 0.64 | 0.63 | 1.12 | 1.11 |
| MIT 9952-280 | 0 | 0 | 0.75 | 0.67 | 1.01 | 1.12 |
| MIT 9952-281 | 0 | 0 | 0.59 | 0.34 | 1.0 | 0.96 |
| MIT 9952-282 | 0 | 0 | 0.49 | 0.5 | 0.82 | 0.89 |
| MIT 9952-283 | 0 | 0 | 0.53 | 0.48 | 0.97 | 1.0 |
| MIT 9952-284 | 0 | 0 | 0.65 | 0.54 | 0.91 | 0.96 |
| MIT 9952-285 | 0 | 0 | 0.57 | 0.53 | 0.9 | 1.07 |
| MIT 9952-286 | 0 | 0 | 0.62 | 0.64 | 0.96 | 1.11 |
| MIT 9952-287 | 0 | | 1.18 | 1.32 | 1.07 | 1.1 |
| MIT 9952-288 | 0 | 0 | 0.59 | 0.52 | 0.77 | 0.77 |
| MIT 9952-289 | 0 | 0 | 0.6 | 0.64 | 1.0 | 0.98 |
| MIT 9952-290 | 0 | 0 | 0.52 | 0.56 | 0.87 | 0.82 |
| MIT 9952-291 | 0 | 0 | 0.55 | 0.51 | 0.94 | 0.97 |
| MIT 9952-292 | 0 | 0 | 0.47 | 0.58 | 1.06 | 1.01 |
| MIT 9952-293 | 0 | 0 | 0.69 | 0.67 | 0.85 | 0.95 |
| MIT 9952-294 | 0 | 0 | 0.61 | 0.56 | 0.93 | 0.95 |
| MIT 9952-295 | 0 | 0 | 0.64 | 0.58 | 1.01 | 0.95 |
| MIT 9952-296 | 0 | 0 | 0.63 | 0.61 | 1.05 | 0.98 |
| MIT 9952-297 | 0 | 0 | 0.56 | 0.46 | 1.07 | 1.09 |
| MIT 9952-298 | 0 | 0 | 1.29 | 1.24 | 1.07 | 1.02 |
| (8^{th}) | | | | | | |
| MIT 9952-299 | 0 | | 0.73 | 0.57 | 1.05 | 0.99 |
| MIT 9952-300 | 0 | | 0.66 | 0.66 | 1.18 | 0.97 |
| MIT 9952-301 | 0 | | 0.71 | 0.7 | 1.01 | 0.98 |
| MIT 9952-302 | 0 | 0 | 0.52 | 0.55 | 0.79 | 0.85 |
| MIT 9952-303 | 0 | | 0.58 | 0.58 | 0.98 | 0.92 |
| MIT 9952-304 | 0 | | 0.35 | 0.44 | 0.86 | 0.93 |
| MIT 9952-305 | 0 | 0 | 0.67 | 0.6 | 1.07 | 1.01 |
| MIT 9952-306 | 0 | | 0.79 | 0.72 | 1.0 | 0.96 |
| MIT 9952-307 | 0 | | 0.69 | 0.63 | 0.95 | 0.85 |
| MIT 9952-308 | 0 | | 0.57 | 0.69 | 0.84 | 0.98 |
| MIT 9952-309 | 0 | | 0.7 | 0.68 | 1.14 | 1.08 |
| MIT 9952-310 | 0 | | 0.97 | 1.11 | 0.96 | 1.01 |
| MIT 9952-311 | 0 | | 0.63 | 0.65 | 0.98 | 0.99 |
| MIT 9952-312 | 0 | | 0.45 | 0.75 | 0.97 | 0.88 |
| MIT 9952-313 | 0 | | 0.79 | 0.77 | 0.94 | 0.98 |
| MIT 9952-314 | 0 | | 0.55 | 0.54 | 1.24 | 0.94 |
| MIT 9952-315 | 0 | 0 | 0.51 | 0.53 | 0.86 | 0.73 |
| MIT 9952-316 | 0 | | 0.71 | 0.72 | 1.13 | 1.1 |
| MIT 9952-317 | 0 | | 0.69 | 0.73 | 1.0 | 0.96 |
| MIT 9952-318 | 0 | | 0.67 | 0.81 | 1.18 | 0.94 |
| MIT 9952-319 | 0 | | 1.13 | 1.17 | 0.89 | 1.07 |
| MIT 9952-320 | 0 | | 0.54 | 0.83 | 1.04 | 1.01 |
| MIT 9952-321 | 0 | | 1.22 | 1.11 | 1.96 | 1.03 |
| MIT 9952-322 | 0 | | 0.79 | 0.86 | 0.1 | 0.96 |
| MIT 9952-323 | 0 | 0 | 0.46 | 0.63 | 0.93 | 0.94 |
| MIT 9952-324 | 0 | 0 | 0.55 | 0.56 | 0.92 | 0.84 |
| MIT 9952-325 | 0 | 0 | 0.56 | 0.49 | 1.05 | 1.05 |
| MIT 9952-326 | 0 | 0 | 0.55 | 0.53 | 0.93 | 0.98 |
| MIT 9952-327 | 0 | 0 | 0.4 | 0.45 | 1.18 | 1.13 |
| MIT 9952-328 | 4 | 4 | 0.5 | 0.56 | 0.93 | 1.12 |
| MIT 9952-329 | 0 | 0 | 0.57 | 0.53 | 1.0 | 0.98 |
| MIT 9952-330 | 0 | 0 | 0.59 | 0.56 | 1.02 | 1.07 |
| MIT 9952-331 | 0 | 0 | 0.63 | 0.35 | 1.05 | 1.0 |
| MIT 9952-332 | 0 | 0 | 0.69 | 0.67 | 1.26 | 1.25 |
| MIT 9952-333 | 0 | 0 | 0.56 | 0.59 | 1.05 | 1.03 |
| MIT 9952-334 | 0 | 0 | 0.57 | 0.55 | 0.95 | 0.98 |
| MIT 9952-335 | 0 | 0 | 0.57 | 0.49 | 1.0 | 0.95 |
| MIT 9952-336 | 0 | 0 | 0.54 | 0.58 | 1.1 | 1.14 |
| MIT 9952-337 | 0 | 0 | 0.6 | 0.57 | 0.92 | 0.96 |
| MIT 9952-338 | 0 | 0 | 0.61 | 0.6 | 1.01 | 1.13 |
| (9^{th}) | | | | | | |
| MIT 9952-339 | 0 | 0 | 0.58 | 0.54 | 0.94 | 0.93 |
| MIT 9952-340 | 0 | 0 | 0.48 | 0.44 | 0.9 | 0.96 |
| MIT 9952-341 | 2 | 2 | 0.61 | 0.51 | 0.99 | 1.01 |
| MIT 9952-342 | 0 | 0 | 0.43 | 0.44 | 0.92 | 0.97 |

Inhibition of Selective Lipid Uptake by BLTs is Specific.

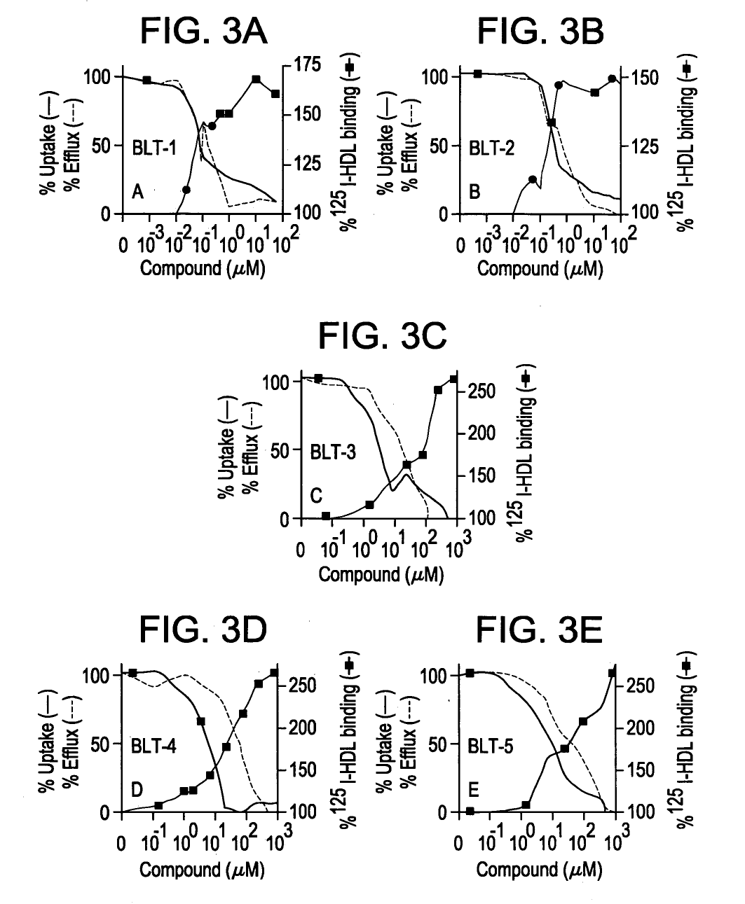

The specificity of BLT inhibition was tested by testing their effects on several other cellular properties at their concentrations that inhibit [³H]CE uptake by 95% (IC_{CE}95) (FIG. 3). None of the BLTs disrupted the integrity of the actin- and tubulin networks. They also did not inhibit the uptake or alter the intracellular distribution of the fluorescently labeled endocytic receptor ligands transferrin and epidermal growth factor. The BLTs also failed to inhibit the uptake of fluorescently labeled cholera toxin from the cell surface to perinuclear regions through a pathway believed to depend in part on cholesterol- and sphingolipid-rich lipid rafts (Lencer, et al. (1999) Biochim. Biophys. Acta 1450, 177-190). Moreover, BLTs did not interfere with the secretory pathway, as assessed by analysis of the transport of the enhanced green fluorescent protein-labeled integral viral membrane glycoprotein VSV G (VSVG^{ts045}-EGFP). Thus, BLTs do not induce general defects in clathrin-dependent and clathrin-independent intracellular membrane trafficking or in the organization of the cytoskeleton and are, by these criteria, specific inhibitors of SR-BI-dependent lipid uptake.

BLTs Inhibit SR-BI-Mediated Cholesterol Efflux from Cells to HDL.

In addition to mediating selective lipid uptake from HDL, SR-BI can facilitate the efflux of unesterified cholesterol from cells to HDL particles (Jian, et al. (1998) J Biol Chem 273, 5599-606. Ji, et al. (1997) J. Biol. Chem. 272, 20982-5). To determine if the BLTs could inhibit this SR-BI-mediated lipid transport activity, cells were labeled with [³H]cholesterol and its efflux to unlabeled HDL measured in the presence or absence of the BLTs. (FIG. 2C, table II). Cells were incubated for 3 hrs in the absence (top panels) or presence (bottom panels) of 50 micromolar BLT-1 (MIT 9952-53) and epifluorescence light microscopy was used to monitor the following cellular activities: clathrin-dependent endocytosis of fluorescently labeled transferrin (A,B; HeLa cells) and EGF (C,D; HeLa cells); clathrin-independent endocytosis of fluorescently labeled cholera toxin (E, F; BSC-1 cells), and transport of the temperature sensitive fluorescent membrane protein VSVG^{ts045}-EGFP from the ER to the cell surface (G,H; BSC-1 cells). In addition, the intracellular distributions of the actin cytoskeleton (visualized with rhodamine labeled phalloidin, I,J; ldlA-[mSRBI] cells) and the tubulin network (visualized with fluorescently labeled antibodies specific to y-tubulin, K,L; BSC-1 cells) were determined. BLT-1 (MIT 9952-53) and the other BLTs (not shown) had no effects on any of these cellular properties or activities.

As shown in Table III, all BLTs inhibited SR-BI-mediated cholesterol efflux with relative potencies (IC_{FC}50s) similar to those for [³H]CE uptake; although in the cases of BLT-3 (MIT 9952-19), BLT-4 (MIT 9952-29) and BLT-5 (MIT 9952-6), the IC_{FC}50s for efflux were higher than those for uptake, suggesting that the BLTs may have uncovered possible differences in the mechanisms of uptake and efflux. The BLTs had little effect on the SR-BI-independent efflux (not inhibited by the specific anti-SR-BI blocking antibody KKB-1) (Kapoor, et al. (2000) Journal of Cell Biology 150, 975-88). In untransfected ldlA-7 cells expressing relatively low levels of endogenous SR-BI, total and SR-BI-dependent (e.g. KKB-1-inhibitable) cholesterol efflux were substantially lower (˜5-10-fold) than in ldlA[mSR-BI] cells. The BLTs were able to inhibit the low SR-BI-dependent cholesterol efflux in ldlA-7 cells, but had no inhibitory effect on the similarly low SR-BI-independent efflux.

TABLE III

| | | 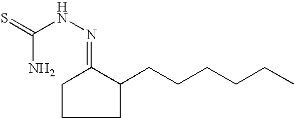 | 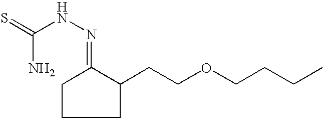 |
|---|---|---|---|
| | | BLT-1 | BLT-2 |
| | n | meant ± SD | meant ± SD |
| (A) EC50 (μM) | | | |
| DiI-HDL uptake | 3 | 0.06 ± 0.04 | 0.35 ± 0.18 |
| [³H]CEt HDL uptake | 6 | 0.11 ± 0.08 | 0.24 ± 0.1 |
| (Y1-BS1 cells) | 2 | 0.38 NA | 0.41 NA |
| [³H]cholesterol efflux | 3 | 0.15 ± 0.09 | 0.47 ± 0.23 |
| ¹ⁿI-HDL binding | 3 | 0.088 ± 0.05 | 0.25 ± 0.13 |
| (B) Binding | | | |
| Parameters | | | |
| apparent K_{d} (μg ml⁻¹) | 3 | 4.7 ± 0.05 | 6.0 ± 6.0 |
| K_{off} (min⁻¹) | 2 | 0.06 NA | 0.062 NA |
| Bmax (%) | | 95.8 ± 10.1 | 93.0 ± 20.5 |
| EC₅₀ (μM) | | | |

| | 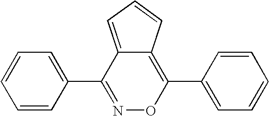 | 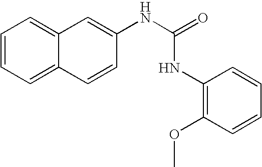 |
|---|---|---|
| | BLT-3 | BLT-4 |
| | meant ± SD | meant ± SD |
| (A) EC50 (μM) | | |
| DiI-HDL uptake | 0.51 ± 0.15 | 2.0 ± 1.0 |
| [³H]CEt HDL uptake | 2.3 ± 1.5 | ¹² 3.9¹ ± 0.76 |
| (Y1-BS1 cells) | 1.7 NA | 4.4 NA |
| [³H]cholesterol efflux | 17.2 ± 4.0 | 54.9 ± 35.2 |
| ¹ⁿI-HDL binding | 46.5 ± 49.3 | 24.9 ± 14.8 |
| (B) Binding | | |
| Parameters | | |
| apparent K_{d} (μg ml⁻¹) | 8.0 ± 4.0 | 8.9 ± 2.3 |
| K_{off} (min⁻¹) | 0.08 NA | 0.082 NA |
| Bmax (%) | 85.8 ± 15.8 | 79.9 ± 15.9 |
| EC₅₀ (μM) | | |

| | 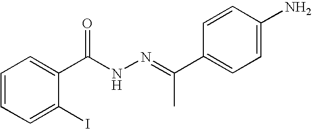 | |
|---|---|---|
| | BLT-5 | No ELT |
| | meant ± SD | meant ± SD |
| (A) EC50 (μM) | | |
| DiI-HDL uptake | 7.1 ± 3.7 | — |
| [³H]CEt HDL uptake | 13.8¹ ± 8.5 ¹¹ | — |
| (Y1-BS1 cells) | 8.0 NA | — |
| [³H]cholesterol efflux | 75.3 ± 40.1 | — |
| ¹ⁿI-HDL binding | 18.0 ± 3.7 | — |
| (B) Binding | | |
| Parameters | | |
| apparent K_{d} (μg ml⁻¹) | 12.0 ± 1.6 | 16.6 ± 1.5 |
| K_{off} (min⁻¹) | 0.079 NA | 0.11 NA |
| Bmax (%) | 92.1 ± 36.8 | 100.0 ± 18.4 |
| EC₅₀ (μM) | | |

¹n = 5
Footnote: all experiments were with ld1A [mSR-BI] cells except where noted.

BLTs Do Not Change the Surface Expression of SR-BI.

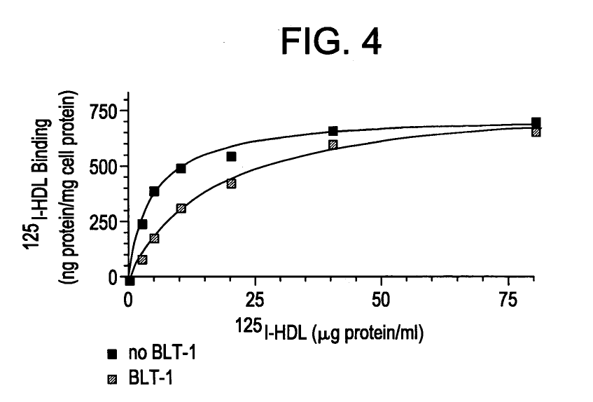

To determine if BLTs inhibited SR-BI function by reducing its cell surface expression, we measured surface expression using the KKB-1 anti-mSR-BI antibody and flow cytometry. FIG. 4 shows that, after a 3 hr incubation at their IC_{CE}95s (corresponding tol AM for BLTs 1 (MIT 9952-53) and 2 (MIT 9952-61), 50 μM for BLTs 3-5 (MIT 9952-19, MIT 9952-29, and MIT 9952-6)), the BLTs did not alter the expression of mSR-BI on the surfaces of ldlA[mSR-BI] cells.

BLTs Enhance Binding of HDL to SR-BI.

It was initially expected that the BLTs would function by inhibiting HDL binding to SR-BI. However, when cells were incubated with a sub-saturating concentration of either [³H]CE-HDL or ¹²⁵I-labeled HDL (¹²⁵I-HDL) (10 μg protein/ml) and increasing amounts of compound (FIG. 5), the decreases in [³H]CE uptake (solid lines, no symbols, data from FIG. 2B) and [3H]cholesterol efflux (dashed lines, data from FIG. 2C) were accompanied by corresponding increases in ¹²⁵I-HDL binding (solid lines, square symbols). The concentration dependence of ¹²⁵I-HDL binding was determined in the presence or absence of BLTs at their IC_{CE}95 concentrations (FIG. 6 and Table II). The BLTs did not substantially alter the number of binding sites (Bₘₐₓ), but rather induced small, yet significant, increases in the affinity of SR-BI for HDL (lower apparent Kds). Furthermore, the BLTs reduced the rates of dissociation of ¹²⁵I-HDL from SR-BI (Table II), indicating that the tighter binding induced by the BLTs was due, at least in part, to a decrease in the dissociation rate.

Discussion

200 compounds, shown in Table I, altering SR-BI mediated lipid transport were identified using in vitro assays. Results of testing are shown in Table II. BLT-1 (MIT 9952-53) through BLT-5 (MIT 9952-6) were identified as small molecules that inhibit the transfer of lipids between HDL and cells mediated by the HDL receptor SR-BI. BLTs inhibited both cellular selective lipid uptake of HDL cholesteryl ether and efflux of cellular cholesterol to HDL. The inhibitory effects of the BLTs were specific (for example, they specifically alter SR-BI binding), as they required the expression of active SR-BI receptors and they did not interfere with several clathrin-dependent and independent endocytic pathways, the secretory pathway nor the actin- or tubulin cytoskeletal networks. Strikingly, inhibition of lipid transfer by BLTs was accompanied by enhanced HDL binding affinity (reduced dissociation rates).

Modifications and variations of the methods and materials described herein will be obvious to those skilled in the art and are intended to be encompassed by the following claims. The teachings of the references cited herein are specifically incorporated herein.

We claim:

1. A compound which specifically alters the binding activity of SR-BI, in combination with a pharmaceutically acceptable carrier, in an effective amount to treat a human or animal in need thereof, obtained by screening a library of compounds for alteration of SR-B1 binding activity or expression.

2. The compound of claim 1 selected from the group shown in Table I.

3. The compound of claim 1, selected from the group consisting of BLT-1 (MIT 9952-53), BLT-2 (MIT 9952-61), BLT-3 (MIT 9952-19), BLT-4 (MIT 9952-29), and BLT-5 (MIT 9952-6).

4. A method for altering cholesterol transport into or out of cells comprising inhibiting expression or activity of SR-BI comprising administering to an animal or human in need thereof the composition of claim 1.

5. The method of claim 4, wherein the composition of claim 1 enhances HDL binding by increasing SR-BI's binding affinity for HDL.

6. The method of claim 4, wherein the inhibited SR-BI binding activity blocks SR-BI-mediated lipid transport.

7. The method of claim 6, wherein the inhibited SR-BI binding activity blocks SR-BI-mediated selective lipid uptake.

8. The method of claim 7, wherein the lipid is HDL cholesteryl ether.

9. The method of claim 4, wherein the inhibited SR-BI binding activity blocks efflux of cellular cholesterol to HDL.

10. A method of identifying a compound which alters SR-BI binding activity or expression comprising screening a library of compounds.

11. The method of claim 10, wherein the SR-BI expression is determined by Northern analysis.

12. The method of claim 10, wherein the library is a chemical library.

13. The method of claim 10, wherein the SR-BI binding activity is inhibited.

14. The method of claim 13, wherein the inhibited SR-BI binding activity blocks SR-BI-mediated lipid transport.

15. The method of claim 14, wherein the inhibited SR-BI binding activity blocks SR-BI-mediated selective lipid uptake.

16. The method of claim 15, wherein the lipid is HDL cholesteryl ether.

17. The method of claim 10, wherein the inhibited SR-BI binding activity blocks efflux of cellular cholesterol to HDL.
